(19) United States
(12) Patent Application Publication
(10) Pub. No.: US 2002/0165142 A1
(43) Pub. Date: Nov. 7, 2002

(54) DAZ genes

(75) Inventors: Richa Saxena, New York, NY (US); David C. Page, Winchester, MA (US); Helen Skaletsky, Milton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA (US)

(21) Appl. No.: 09/917,286

(22) Filed: Jul. 27, 2001

Related U.S. Application Data
(60) Provisional application No. 60/221,065, filed on Jul. 27, 2000.

(51) Int. Cl. A61K 38/17; C07H 21/04; C12P 21/02; C12N 5/06; C07K 14/715

(52) U.S. Cl. 514/12; 530/350; 435/69.1; 435/320.1; 435/325; 536/23.5

(57) ABSTRACT

Four DAZ genes in the AZFc region of the human Y chromosome are disclosed. Methods of using the disclosed genes and gene products are described, along with methods and reagents to distinguish between the DAZ genes are also described.

### RELATED APPLICATIONS

This application claims the benefit of U.S. Provisional Application No. 60/221,065, filed Jul. 27, 2000, the entire teachings of which are incorporated herein by reference.

### GOVERNMENT SUPPORT

The invention was supported, in whole or in part, by Grant HD-32907 from the National Institutes of Health. The Government has certain rights in the invention.

### BACKGROUND OF THE INVENTION

Approximately two percent of men are infertile because they produce few or no sperm (Silber, (1989) Hum. Reprod. 4:947-953). The most common known molecular cause of such spermatogenic failure is deletion of the AZFc region on the long arm of the human Y chromosome (Ma, 1992; Reijo, 1995; Vogt, 1996). Although one or more spermatogenesis genes must lie within the AZFc region, the identity of the critical factor(s) is still uncertain because no point mutations or internal deletions in candidate genes have been identified. Candidate genes within this region include DAZ, BPY2, RBMY, and CDY1 (Reijo, 1995; Lahn and Page, 1997; Yen, 1998).

The DAZ (Deleted in Azoospermia) genes, which encode putative RNA-binding proteins, are strong AZFc candidates. The DAZ genes are located exclusively within the AZFc region and are transcribed only in testicular germ cells (Reijo, 1995; Saxena, 1996; Menke, 1997). In model organisms, genetic studies have demonstrated that DAZ play essential roles in germ cell development (Eberhart, 1996; Ruggiu, 1997; Houston and King, 2000). In mice, disruption of the Dazl gene leads to germ cell loss before birth, rendering both males and females infertile (Ruggiu, 1997). In Drosophila, males mutant for the DAZ homolog boule are infertile with germ cell arrest at the G2/M transition into meiosis (Eberhart, 1996).

The precise number of DAZ genes in the AZFc region has been difficult to determine. Initially, only one DAZ gene was thought to exist within the AZFc region (Reijo, 1995). However, it was determined that DAZ cosmids derived from a single individual differed slightly in DNA sequence, providing evidence for at least two distinct DAZ genes (Saxena, 1996). Fluorescence in situ hybridization provided evidence of multiple DAZ genes on Yq (Glaser, 1997), while Southern blotting and long-range restriction mapping provide evidence of at least three DAZ genes on the Y chromosome (Yen, 1997; Yen, 1998). Most recently, fiber-FISH analysis provided evidence of seven DAZ genes or pseudogenes on Yq (Glaser, 1989).

Thus, not only is the number of DAZ genes on the Y chromosome unknown, it is not known which of the potential genes are functional and whether some of the DAZ genes are in fact pseudogenes. Furthermore, genes on Y chromosomes are often subject to degeneration during evolution (Ohno, 1967; Rice, 1994; Charlesworth, 1996). Repetitive gene families on the human Y chromosome may include both functional and corrupted gene copies. Therefore, it is not clear whether the DAZ gene faimly would include some transcriptionally active and some decayed family members. It would be useful for the diagnosis of DAZ-related dysfunctions, treatment of said dysfunctions, and research involving DAZ genes to determine the gene structure of DAZ and the number of functional DAZ genes. Further, there is a need for a method to distinguish the DAZ genes and pseudogenes from each other.

### SUMMARY OF THE INVENTION

As a result of the present invention, the number of DAZ genes on the Y chromosome has been determined along with the number of transcribed genes. Despite the degeneration of DAZ genes 1-4 s described herein, the cDNA sequence of three of those genes, DAZ2-4, is also provided. As further described herein, the invention provides methods and reagents to distinguish between the DAZ genes, and as a result it is possible to analyze DAZ gene transcription and DAZ gene products and to distinguish among them.

The present invention is directed to isolated nucleic acid molecules comprising DAZ gene cDNA. The isolated nucleic acid molecules of the present invention comprise at least one nucleotide sequence selected from the group consisting of SEQ ID NOS: 1, 3, 5, 7, 9, 10, 11, 12, 13, 14, 15, 16, 17, 18, 19, 20, 21, 22 or 23 and their complements. In one embodiment, the nucleic acid molecules of the present invention comprise a nucleotide sequence which is at least about 60% identical to a nucleotide sequence selected from the group consisting of SEQ ID NOS: 1, 3, 5, 7, 9-23 and their complements. In another embodiment, the invention relates to nucleic acid molecules comprising a nucleotide sequence that hybridizes to a nucleotide sequence selected from the group consisting of SEQ ID NOS: 1, 3, 5, 7, 9-23 and their complements under conditions of high stringency. The present invention also relates to an isolated nucleic acid molecule which encodes SEQ ID NO: 2, SEQ ID NO: 4, SEQ ID NO: 6 or SEQ ID NO: 8.

The present invention further relates to DAZ polypeptides. In one embodiment, the DAZ polypeptide comprises SEQ ID NO: 2, SEQ ID NO: 4, SEQ ID NO: 6 or SEQ ID NO: 8 or functional (e.g., antigenic) fragments thereof. In another embodiment, the DAZ polypeptide comprises the amino acid sequence encoded by SEQ ID NO: 1, SEQ ID NO: 3, SEQ ID NO: 5 or SEQ ID NO: 7.

The present invention also relates to antibodies specific for DAZ polypeptides, or antigen-binding fragments thereof, which selectively bind to a polypeptide comprising SEQ ID NO: 2, SEQ ID NO: 4, SEQ ID NO: 6 or SEQ ID NO: 8 or functional (e.g., antigenic) fragments thereof.

The present invention is also directed to methods for assaying for the presence of a DAZ polypeptide or portion thereof in a sample. The method comprises contacting the sample with an agent (e.g., an antibody) which specifically detects the DAZ polypeptide. In one embodiment, the DAZ polypeptide is encoded by SEQ ID NO: 1, 3, 5 or 7. In another embodiment, the DAZ polypeptide comprises the amino acid sequence of SEQ ID NO: 2, 4, 6 or 8. In one embodiment, the method comprises contacting the sample with an antibody, wherein the antibody specifically binds to the DAZ polypeptide, and detecting bound antibody.

The present invention is further directed to a method of assaying for the presence of a DAZ nucleic acid molecule in a sample. The method comprises contacting the sample with a nucleotide sequence selected from the group consisting of SEQ ID NOS: 1, 3, 5, 7, 9-23, a portion of any one of said sequences which is at least 10 nucleotides in length, or complements thereof, under conditions appropriate for selective hybridization, such that the nucleotide sequence binds to complementary nucleic acid molecule, if present, in the sample. The hybridized nucleotide sequence (e.g., the complex) is then detected, thereby assaying for the presence of a DAZ nucleic acid molecule in a sample.

The present invention is also drawn to a method for distinguishing a DAZ gene of interest from other DAZ genes by detecting sequence family variants. The method comprises conducting at least one amplification reaction to amplify at least one region of a DAZ gene; digesting the amplified product with a restriction endonuclease; and detecting products of the digestion, wherein the products of the digestion distinguish the DAZ gene of interest from other DAZ genes.

The present invention is also drawn to methods of increasing or reducing (inhibiting) DAZ gene expression in a cell. In one embodiment, DAZ gene expression in a cell is reduced by a method comprising contacting the cell with a polynucleic acid complementary to at least about 20 contiguous nucleotides of the DAZ gene. For example, the 20 contiguous nucleotides can be a polynucleotide sequence selected from the group consisting of about nucleotide 197 to about nucleotide 1873 of SEQ ID NO: 1, about nucleotide 189 to about nucleotide 1649 of SEQ ID NO: 3, and about nucleotide 1 to about nucleotide 1242 of SEQ ID NO: 5, such that the polynucleic acid enters the cell in sufficient quantity to bind to DAZ gene mRNA, thereby triggering destruction of the bound DAZ gene mRNA and reducing DAZ gene expression in the cell.

In another embodiment, DAZ gene expression is increased in a cell. The method comprises contacting the cell with a DAZ nucleic acid molecule. For example, the nucleic acid molecules can comprise a sequence selected from the group consisting of from about nucleotide 197 to about nucleotide 1873 of SEQ ID NO: 1, from about nucleotide 189 to about nucleotide 1649 of SEQ ID NO: 3, and from about nucleotide 1 to about nucleotide 1242 of SEQ ID NO: 5. The nucleic acid molecule enters the cell and is transcribed, thereby increasing DAZ gene product expression in the cell.

Thus, this invention has application to several areas. It may be used diagnostically to identify males with reduced sperm count in whom a DAZ gene has been altered. It may also be used therapeutically in gene therapy treatments to remedy fertility disorders associated with alteration of a DAZ gene. The present invention may also be useful in designing or identifying agents which function as a male contraceptive by inducing reduced sperm count. This invention also has application as a research tool, as the nucleotide sequences described herein have been localized to the AZFc region of the human Y chromosome and can therefore serve as markers for this region.

### BRIEF DESCRIPTION OF THE DRAWINGS

FIG. 1 is a schematic diagram of the inverted duplication in cosmid 18E8.

FIG. 2 shows genomic organization of four DAZ genes in two clusters as inferred from analysis of BAC and cosmid clones.

FIG. 3 shows a gel analysis of SFVs in DAZ BAC clones scored by PCR-restriction digest analysis.

FIG. 4A shows a Southern blot of a 2.4-kb repeat probe pDP1649 to TaqI-digested DAZ BAC and cosmid DNAs.

FIG. 4B shows a Southern blot of a PCR fragment spanning DAZ exons 2 and 3 to MluI-digested DAZ BAC DNAs.

FIG. 4C is a schematic diagram of 5′ portions of DAZ1 and DAZ2 genes with three tandem copies or one copy, respectively of the 10.8-kb repeat (large open arrow).

FIG. 5 is a schematic of the predicted human DAZL (autosomal) and DAZ (Y-linked) proteins.

FIG. 6 a schematic showing an evolutionary model to account for four DAZ genes in two clusters on the human Y chromosome.

FIG. 7 is a table showing the PCR/restriction digest typing of sequence family variants that distinguish between DAZ genes.

FIGS. 8A and 8B show the nucleotide (SEQ ID NO: 1) and amino acid (SEQ ID NO: 2) sequences of DAZ2.

FIG. 9 shows the nucleotide (SEQ ID NO: 3) and amino acid (SEQ ID NO: 4) sequences of DAZ3.

FIG. 10 shows the partial nucleotide (SEQ ID NO: 5) and amino acid (SEQ ID NO: 6) sequences of DAZ4.

FIG. 11 shows the partial nucleotide (SEQ ID NO: 7) and amino acid (SEQ ID NO: 8) sequences of DAZ4.

FIGS. 12A-12B shows the nucleotide sequences of SEQ ID NOS: 1, 3, 5 and 7 and the amino acid sequences of SEQ ID NOS: 2, 4, 6 and 8.

FIGS. 13A-13C show the protocol for identification of the human STSs derived from Y chromosome genomic clones, as well as the protocol and primers (SEQ ID NOS: 9-23) for identification of four DAZ genes in two clusters found in the AZFc region of the human Y chromosome.

### DETAILED DESCRIPTION OF THE INVENTION

As described herein, FSH analysis and studies of BACs indicate that the human Y chromosome as found in the collection of unrelated individuals studied contains four DAZ genes arranged in two clusters.

Distinguishing among and unambiguously identifying each of the four DAZ genes was technically challenging. As described herein, it was determined that DAZ1, DAZ2, DAZ3, and DAZ4 possess different numbers of intragenic (2.4-kb and 10.8-kb) tandem repeats, but these differences were of little practical use in identifying individual DAZ genes, for several reasons. First, both the 2.4-kb and 10.8-kb repeat arrays were far too large to allow PCR amplification across them (as one might do in the case of mini- or micro-satellites). Second, the DNA sequences of the 10.8-kb repeats appear to be identical one to another obstructing efforts to distinguish among and thereby count the 10.8-kb repeats. Third, many of the BAC clones studied contained portions of two different DAZ genes, further confounding gel-based analyses. Apart from these tandem intragenic amplifications, the DNA sequences of the four DAZ genes appear to be >99.9% identical (Saxena et al. 1996). Consequently, conventional STS-content mapping and restriction fingerprinting of BACs were of little use in distinguishing among the four DAZ genes.

In the end, individual DAZ genes were identified primarily based on subtle sequence differences—especially base-pair substitutions (FIG. 7)— that had been revealed by extensive genomic sequencing (Saxena et al., 1996). Since these subtle differences are among members of a gene family on a single Y chromosome they are not true polymorphisms (which pertain to alleles on homologous chromosomes). We suggest the term “sequence family variants,” or “SFVs” to refer to subtle variation (for example, single nucleotide variation or dinucleotide repeat length variation) between closely related but nonallelic sequences. Based on our experience with the DAZ genes, we anticipate that SFVs will play a crucial role in structural and functional analysis of other segments of the human genome that contain families of closely related sequences.

Based on the genomic and cDNA sequence analysis described herein, at least three Y-chromosomal DAZ genes—DAZ2, DAZ3 and DAZ4— are transcribed and spliced to encode proteins with one or more RRM (RNA recognition motif) domains. As judged by genomic DNA sequence analysis, the remaining Y-chromosomal DAZ gene, DAZ1, is also intact. The DAZ1 coding region is predicted to be the longest of the four genes (744 aa); it is difficult to capture the entire coding region in a single cDNA clone. This problem is compounded by the likelihood that the 5′ portion of the DAZ1 coding region consists of a perfect tandem triplication of a 495-nucleotide, RRM-encoding unit that is duplicated in DAZ4. Finally, the array of exon 7 repeats that is predicted to occur in DAZ1 transcripts is very similar to that observed in DAZ4. Thus, our failure to identify a DAZ1 cDNA clone should not be taken as evidence that DAZ1 is a pseudogene.

The present data also underscore the role of exon pruning during the evolution of the human DAZ genes. As recognized previously, most of the 2.4-kb repeats in human DAZ genes contain a “pseudoexon,” a degenerate, vestigial exon that appears to be excised (as a component of an intron) during processing of DAZ transcripts (Saxena et al., 1996). As diagramed in FIG. 4C, each of the 10.8-kb repeats in human DAZ1 and DAZ4 contains three pseudoexons. Thus, not only the 2.4-kb repeat arrays but also the 10.8-kb repeat arrays appear to be riddled with pseudoexons, at least in humans. In all, th four DAZ genes on the human Y chromosome studied here appear to possess a total of 96 exons and 66 pseudoexons. By contrast, their autosomal progenitor, DAZL, is a conventionally structured gene with only 11 exons. Remarkably, the reading frames of the Y-chromosomal DAZ genes emerged intact from the bouts of intragenic amplification and exon pruning that evidently occurred during evolution. The preserved reading frames suggest that selective pressure on the DAZ proteins was maintained during the evolution of the human DAZ genes.

An evolutionary model is shown in FIG. 6. Following transposition, to the Y chromosome, the ancestral DAZ gene underwent amplification of the 2.4-kb and 10.8-kb units and pruning of many exons. Exons 1, 10, and 11 of each gene are shown. The 2.4-kb repeat unit is represented by a small arrowhead. The 10.8-kb repeat unit is represented by the larger open arrow. A THE element between the inverted DAZ genes is shown as a small black box.

Thus, the present invention is drawn to isolated DAZ nucleic acid molecules or portions thereof and isolated DAZ polypeptides or fragments thereof encoded by said DNA. The present invention is also drawn to antibodies specific for DAZ polypeptides, or antigen-binding fragments thereof. The nucleic acids, proteins and antibodies of the present invention can be used to detect DAZ genes, gene transcripts or gene products. Further, the nucleic acids of the present invention can be used to increase or decrease DAZ transcription in a cell. The present invention is also drawn to methods and reagents for distinguishing one DAZ gene from other DAZ genes.

In one embodiment, the isolated nucleic acid molecules of the present invention comprise one or more of the nucleotide sequences of SEQ ID NOS: 1, 3, 5, 7, 9, 10, 11, 12, 13, 14, 15, 16, 17, 18, 19, 20, 21, 22 or 23 and their complements. In another embodiment, the nucleic acid molecules of the present invention comprise a nucleotide sequence which is at least about 60% identical, more preferably greater than about 75 % identical, and even more preferably greater than about 90% identical to a nucleotide sequence of SEQ ID NOS: 1, 3, 5, 7, 9, 10, 11, 12, 13, 14, 15, 16, 17, 18, 19, 20, 21, 22 or 23 and their complements. In another embodiment, the nucleic acid molecules of the present invention comprise a nucleotide sequence that hybridizes to a nucleotide sequence of SEQ ID NO: 1, 3, 5, 7, 9, 10, 11, 12, 13, 14, 15, 16, 17, 18, 19, 20, 21, 22 or 23 and their complements under conditions of high stringency. In a preferred embodiment, the nucleic acid molecule which hybridizes under conditions of high stringency selected from the group consisting of SEQ ID NO: 1, 3, 5, 7, 9, 10, 11, 12, 13, 14, 15, 16, 17, 18, 19, 20, 21, 22 or 23 and their complements are isolated from human tissue. The present invention also includes an isolated nucleic acid molecule comprising the nucleic acid molecule encoding SEQ ID NO: 2, SEQ ID NO: 4 or SEQ ID NO: 6.

The invention also relates to an isolated nucleic acid molecule consisting of a nucleotide sequence selected from the group consisting of SEQ ID NO: 1, 3, 5, 7, 9, 10, 11, 12, 13, 14, 15, 16, 17, 18, 19, 20, 21, 22 or 23 and the complement of SEQ ID NO: 1, 3, 5, 7, 9, 10, 11, 12, 13, 14, 15, 16, 17, 18, 19, 20, 21, 22 or 23. The invention further relates to an isolated portion of any of SEQ ID NO: 1, 3, 5, 7, 9, 10, 11, 12, 13, 14, 15, 16, 17, 18, 19, 20, 21, 22 or 23 and the complement of SEQ ID NO: 1, 3, 5, 7, 9, 10, 11, 12, 13, 14, 15, 16, 17,18, 19, 20, 21, 22 or 23, which portion is sufficient in length to distinctly characterize the sequence. For example, the isolated portion can be from about 7 to about 15 nucleotides in length, preferably from about 10 to about 20 nucleotides in length, and more preferably from about 15 to about 25 nucleotides in length.

As appropriate, nucleic acid molecules of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense, strand. Preferably, the nucleic acid molecule comprises at least about 10 nucleotides, more preferably at least about 50 nucleotides, and even more preferably at least about 200 nucleotides. The nucleic acid molecule can include all or a portion of the coding sequence of a gene and can further comprise additional non-coding sequences such as introns and non-coding 3′ and 5′ sequences (including regulatory sequences, for example). Additionally, the nucleic acid molecule can be fused to a marker sequence, for example, a sequence which encodes a polypeptide to assist in isolation or purification of the polypeptide. Such sequences include, but are not limited to, those which encode a glutathione-S-transferase (GST) fusion protein and those which encode a hemaglutin A (HA) polypeptide marker from influenza.

As used herein, an “isolated” gene or nucleic acid molecule is intended to mean a gene or nucleic acid molecule which is not flanked by nucleic acid molecules which normally (in nature) flank the gene or nucleic acid molecule (such as in genomic sequences) and/or has been completely or partially purified from other transcribed sequences (as in a cDNA or RNA library). For example, an isolated nucleic acid of the invention may be substantially isolated with respect to the complex cellular milieu in which it naturally occurs. In some instances, the isolated material will form part of a composition (for example, a crude extract containing other substances), buffer system or reagent mix. In other circumstance, the material may be purified to essential homogeneity, for example as determined by PAGE or column chromatography such as HPLC. Preferably, an isolated nucleic acid comprises at least about 50, 80 or 90 percent (on a molar basis) of all macromolecular species present. Thus, an isolated gene or nucleic acid molecule can include a gene or nucleic acid molecule which is synthesized chemically or by recombinant means. Thus, recombinant DNA contained in a vector are included in the definition of “isolated” as used herein. Also, isolated nucleic acid molecules include recombinant DNA molecules in heterologous host cells, as well as partially or substantially purified DNA molecules in solution. In vivo and in vitro RNA transcripts of the DNA molecules of the present invention are also encompassed by “isolated” nucleic acid molecules. Such isolated nucleic acid molecules are useful in the manufacture of the encoded protein, as probes for isolating homologous sequences (e.g., from other mammalian species), for gene mapping (e.g., by in situ hybridization with chromosomes), or for detecting expression of the gene in tissue (e.g., human tissue such as testis tissue), such as by Northern blot analysis.

The invention described herein also relates to fragments or portions of the isolated nucleic acid molecules described above. The term “fragment” is intended to encompass a portion of a nucleic acid molecule described herein which is from at least about 7 contiguous nucleotides to at least about 25 contiguous nucleotides or longer in length; such fragments are useful as probes, e.g., for diagnostic methods and also as primers. Particularly preferred primers and probes selectively hybridize to nucleic acid molecules comprising the nucleotide sequences of any of SEQ ID NO: 1, 3, 5, 7, 9, 10, 11, 12, 13, 14, 15, 16, 17, 18, 19, 20, 21, 22 or 23 and the complement of SEQ ID NO: 1, 3, 5, 7, 9, 10, 11, 12, 13, 14, 15, 16, 17, 18, 19, 20, 21, 22 or 23. The probes and primers can be any length, provided that they are of sufficient length and appropriate composition (i.e., appropriate nucleotide sequence) to hybridize to all or an identifying or characteristic portion of the gene described or to a disrupted form of the gene, and remain hybridized under the conditions used. Useful probes include, but are not limited to, nucleotide sequences which distinguish between the DAZ gene and an altered form of the DAZ gene shown, as described herein, to be associated with reduced sperm count (azoospermia, oligospermia).

The present invention is further directed to a method of assaying for the presence of a nucleic acid molecule of interest in a sample. The method comprises contacting the sample with a nucleotide sequence selected from the group consisting of SEQ ID NOS: 1, 3, 5, 7, 9-23, a portion of any one of said sequences which is at least 10 nucleotides in length, or complements thereof under conditions appropriate for selective hybridization, such that the nucleotide sequence binds to complementary nucleic acid molecule, if present, in the sample. The hybridized nucleotide sequence is then detected, thereby assaying for the presence of a nucleic acid molecule of interest in a sample. In one embodiment, a region of the gene of interest comprising intron 3 is amplified using primers comprising SEQ ID NOS: 9 and 10. The resulting product is digested with Sau3A and the digestion separated by size. DAZ1 and DAZ4 are revealed by the presence from polynucleotide fragments of 63 and 189 base pairs in length. DAZ2 and DAZ3 are revealed by the presence of polynucleotide fragments of 59, 63 and 130 base pairs in length. In another embodiment, a region of the gene of interest comprising intron 6 is amplified using primers comprising SEQ ID NOS: 12 and 13. The resulting product is digested with TaqI and the digestion is separated by size. DAZ1, DAZ3 and DAZ4 are revealed by the presence of polynucleotide fragments of 117 and 184 base pairs in length. DAZ2 is revealed by the presence of a polynucleotide fragement of 301 base pairs in length. In another embodiment, a region of the gene of interest comprising intron 10 is amplified using primers comprising SEQ ID NOS: 15 and 16 The resulting product is digested with DraI and the digestion is separated by size. DAZ1 and DAZ2 are revealed by the presence of polynucleotide fragments of 26, 49, 73 and 122 base pairs in length. DAZ3 and DAZ4 are revealed by the presence of a polynucleotide fragments of 26, 49 and 195 base pairs in length. Thus in one embodiment, the amplification of the gene of interest of portion thereof is conducted by polymerase chain reaction using primers selected from the group consisting of SEQ ID NOS: 9, 10, 12, 13, 15, 16, 18, 19, 21, 22, 23, 24 and combinations thereof. The amplified product can be digested with a restriction enzyme selected from the group consisting of Sau3A, TaqI, DraI and combinations thereof.

The invention also pertains to nucleic acid molecules which hybridize under high stringency hybridization conditions (e.g., for selective hybridization) to a nucleotide sequence described herein. Hybridization probes are oligonucleotides which bind in a base-specific manner to a complementary strand of nucleic acid. Such probes include polypeptide nucleic acids, as described in Nielsen et al., Science 254, 1497-1500 (1991). Appropriate stringency conditions are known to those skilled in the art or can be found in standard texts such as Current Protocols in Molecular Biology, John Wiley & Sons, N.Y. (1989), 6.3.1-6.3.6. For example, stringent hybridization conditions include a salt concentration of no more than 1 M and a temperature of at least 25° C. In one embodiment, conditions of 5× SSPE (750 mM NaCl, 50 mM NaPhosphate, 5 mM EDTA, pH 7.4) and a temperature of 25-30° C., or equivalent conditions, are suitable for specific probe hybridizations. Equivalent conditions can be determined by varying one or more of the parameters given as an example, as known in the art, while maintaining a similar degree of identity or similarity between the target nucleic acid molecule and the primer or probe used. Hybridizable nucleic acid molecules are useful as probes and primers for diagnostic applications.

As used herein, the term “primer” refers to a single-stranded oligonucleotide which acts as a point of initiation of template-directed DNA synthesis under appropriate conditions (e.g., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

Accordingly, the invention pertains to nucleic acid molecules which have a substantial identity with the nucleic acid molecules described herein; particularly preferred are nucleic acid molecules which have at least about 90%, and more preferably at least about 95% identity with nucleic acid molecules described herein. Thus, DNA molecules which comprise a sequence which is different from the naturally-occurring nucleic acid molecule but which, due to the degeneracy of the genetic code, encode the same protein or polypeptide are the subject of this invention. The invention also encompasses variations of the nucleic acid molecules of the invention, such as those encoding portions, analogues or derivatives of the encoded protein or polypeptide. Such variations can be naturally-occurring, such as in the case of allelic variation, or non-naturally-occurring, such as those induced by various mutagens and mutagenic processes. Intended variations include, but are not limited to, addition, deletion and substitution of one or more nucleotides which can result in conservative or non-conservative amino acid changes, including additions and deletions. Preferably, the nucleotide or amino acid variations are silent; that is, they do not alter the characteristics or activity of the encoded protein or polypeptide. As used herein, activities of the encoded protein or polypeptide include, but are not limited to, catalytic activity, binding function, antigenic function and oligomerization function.

The nucleotide sequences of the nucleic acid molecules described herein, e.g., SEQ ID NO: 1, 3, 5, 7, 9, 10, 11, 12, 13, 14, 15, 16, 17, 18, 19, 20, 21, 22 or 23 and the complement of SEQ ID NO: 1, 3, 5, 7, 9, 10, 11, 12, 13, 14, 15, 16, 17, 18, 19, 20, 21, 22 or 23, can be amplified by methods known in the art. For example, this can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be radiolabelled and used as a probe for screening a cDNA library derived from testes tissue, e.g., human testes tissue, mRNA in λzap express, ZIPLOX or other suitable vector. Corresponding clones can be isolated, DNA can obtained following in vivo excision, and the cloned insert can be sequenced in either or both orientations by art recognized methods, to identify the correct reading frame encoding a protein of the appropriate molecular weight. For example, the direct analysis of the nucleotide sequence of nucleic acid molecules of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the protein(s) and the DNA encoding the protein can be isolated, sequenced and further characterized.

With respect to protein or polypeptide identification, bands identified by gel analysis can be isolated and purified by HPLC, and the resulting purified protein can be sequenced. Alternatively, the purified protein can be enzymatically digested by methods known in the art to produce polypeptide fragments which can be sequenced. The sequencing can be performed, for example, by the methods of Wilm et al. (Nature 379(6564):466-469 (1996)). The protein may be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

In addition to substantially full-length polypeptides encoded by nucleic acid molecules described herein, and the polypeptides of SEQ ID NOS: 2, 4 or 6, the present invention includes biologically active fragments of the polypeptides, or analogs thereof, including organic molecules which simulate the interactions of the polypeptides. Biologically active fragments include any portion of the full-length polypeptide which confers a biological function on the variant gene product, including ligand binding, and antibody binding. Ligand binding includes binding by nucleic acids, proteins or polypeptides, small biologically active molecules, or large cellular structures.

This invention also pertains to an isolated protein or polypeptide encoded by the nucleic acid molecules of the invention and to the polypeptides comprising SEQ ID NOS: 2, 4 or 6. The encoded proteins or polypeptides of the invention can be partially or substantially purified (e.g., purified to homogeneity), and/or are substantially free of other proteins. According to the invention, the amino acid sequence of the polypeptide can be that of the naturally-occurring protein or can comprise alterations therein. Such alterations include conservative or non-conservative amino acid substitutions, additions and deletions of one or more amino acids; however, such alterations should preserve at least one activity of the encoded protein or polypeptide, i.e., the altered or mutant protein should be an active derivative of the naturally-occurring protein. For example, the mutation(s) can preferably preserve the three dimensional configuration of the binding and/or catalytic site of the native protein. The presence or absence of biological activity or activities can be determined by various functional assays as described herein. Moreover, amino acids which are essential for the function of the encoded protein or polypeptide can be identified by methods known in the art. Particularly useful methods include identification of conserved amino acids in the family or subfamily, site-directed mutagenesis and alanine-scanning mutagenesis (for example, Cunningham and Wells, Science 244:1081-1085 (1989)), crystallization and nuclear magnetic resonance. The altered polypeptides produced by these methods can be tested for particular biologic activities, including immunogenicity and antigenicity.

Specifically, appropriate amino acid alterations can be made on the basis of several criteria, including hydrophobicity, basic or acidic character, charge, polarity, size, the presence or absence of a functional group (e.g., —SH or a glycosylation site), and aromatic character. Assignment of various amino acids to similar groups based on the properties above will be readily apparent to the skilled artisan; further appropriate amino acid changes can also be found in Bowie et al. (Science 247:1306-1310(1990)).

The encoded polypeptide can also be a fusion protein comprising all or a portion of the amino acid sequence fused to an additional component. Additional components, such as radioisotopes and antigenic tags, can be selected to assist in the isolation or purification of the polypeptide or to extend the half life of the polypeptide; for example, a hexahistidine tag would permit ready purification by nickel chromatography. Furthermore, polypeptides of the present invention can be progenitors of the active protein; progenitors are molecules which are cleaved to form an active molecule.

Polypeptides described herein can be isolated from naturally-occurring sources, chemically synthesized or recombinantly produced. Polypeptides or proteins of the present invention can be used as a molecular weight marker on SDS-PAGE gels or on molecular sieve gel filtration columns using art-recognized methods.

The invention also provides expression vectors containing a nucleic acid sequence described herein, operably linked to at least one regulatory sequence. Many such vectors are commercially available, and other suitable vectors can be readily prepared by the skilled artisan. “Operably linked” is intended to meant that the nucleic acid molecule is linked to a regulatory sequence in a manner which allows expression of the nucleic acid sequence. Regulatory sequences are art-recognized and are selected to produce the encoded polypeptide or protein. Accordingly, the term “regulatory sequence” includes promoters, enhancers, and other expression control elements which are described in Goeddel, Gene Expression Technology: Methods in Enzymology 185, Academic Press, San Diego, Calif. (1990). For example, the native regulatory sequences or regulatory sequences native to the transformed host cell can be employed. It should be understood that the design of the expression vector may depend on such factors as the choice of the host cell to be transformed and/or the type of protein desired to be expressed. For instance, the polypeptides of the present invention can be produced by ligating the cloned gene, or a portion thereof, into a vector suitable for expression in either prokaryotic cells, eukaryotic cells or both (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17). Typically, expression constructs will contain one or more selectable markers, including, but not limited to, the gene that encodes dihydrofolate reductase and the genes that confer resistance to neomycin, tetracycline, ampicillin, chloramphenicol, kanamycin and streptomycin resistance.

Prokaryotic and eukaryotic host cells transfected by the described vectors are also provided by this invention. For instance, cells which can be transfected with the vectors of the present invention include, but are not limited to, bacterial cells such as E. coli (e.g., E. coli K12 strains, Streptomyces, Pseudomonas, Serratia marcescens and Salmonella typhimurium, insect cells (baculovirus), including Drosophila, fungal cells, such as yeast cells, plant cells and mammalian cells, such as thymocytes, Chinese hamster ovary cells (CHO), and COS cells.

Thus, a nucleic acid molecule comprising SEQ ID NO: 1, 3, 5, 7, 9, 10, 11, 12, 13, 14, 15, 16, 17, 18, 19, 20, 21, 22 or 23, or a nucleic acid molecule which encodes SEQ ID NO: 2, 4 or 6 as described herein, can be used to produce a recombinant form of the protein via microbial or eukaryotic cellular processes. Ligating the polynucleic acid molecule into a gene construct, such as an expression vector, and transforming or transfecting into hosts, either eukaryotic (yeast, avian, insect, plant or mammalian) or prokaryotic (bacterial cells), are standard procedures used in producing other well known proteins. Similar procedures, or modifications thereof, can be employed to prepare recombinant proteins according to the present invention by microbial means or tissue-culture technology. Accordingly, the invention pertains to the production of encoded proteins or polypeptides by recombinant technology.

The proteins and polypeptides of the present invention can be isolated or purified (e.g., to homogeneity) from recombinant cell culture by a variety of processes. These include, but are not limited to, anion or cation exchange chromatography, ethanol precipitation, affinity chromatography and high performance liquid chromatography (HPLC). The particular method used will depend upon the properties of the polypeptide and the selection of the host cell; appropriate methods will be readily apparent to those skilled in the art.

The present invention also relates to antibodies which bind a polypeptide or protein encoded by SEQ ID NO: 2, 4, 6 or 8, or antigenic fragments thereof. For instance, polyclonal and monoclonal antibodies, including non-human and human antibodies, humanized antibodies, chimeric antibodies and antigen-binding fragments thereof (Current Protocols in Immunology, John Wiley & Sons, N.Y. (1994); EP Application 173,494 (Morrison); International Patent Application W086/01533 (Neuberger); and U.S. Pat. No. 5,225,539 (Winters)) which bind to the described protein or polypeptide are within the scope of the invention. A mammal, such as a mouse, rat, hamster, goat or rabbit, can be immunized with an immunogenic form of the protein (e.g., the full length protein or a polypeptide comprising an antigenic fragment of the protein which is capable of eliciting an antibody response). Techniques for conferring immunogenicity on a protein or polypeptide include conjugation to carriers or other techniques well known in the art. The protein or polypeptide can be administered in the presence of an adjuvant. The progress of immunization can be monitored by detection of antibody titers in plasma or serum. Standard ELISA or other immunoassays can be used with the immunogen as antigen to assess the levels of antibody.

Following immunization, anti-peptide antisera can be obtained, and if desired, polyclonal antibodies can be isolated from the serum. Monoclonal antibodies can also be produced by standard techniques which are well known in the art (Kohler and Milstein, Nature 256:495-497 (1975); Kozbar et al., Immunology Today 4:72 (1983); and Cole et al, Monoclonal Antibodies and Cancer Therapy, Alan R. Liss, Inc., pp. 7796 (1985)). The term “antibody” as used herein is intended to include fragments thereof, such as Fab and F(ab)₂. Antibodies described herein can be used to inhibit the activity of the polypeptides and proteins described herein, particularly in vitro and in cell extracts, using methods known in the art. Additionally, such antibodies, in conjunction with a label, such as a radioactive label, can be used to assay for the presence of the expressed protein in a sample from, e.g., a tissue sample, wherein the antibody specifically binds to the polypeptide encoded by SEQ ID NO: 2, 4, or 6 or antigenic portion thereof and the complex is detected. Such antibodies can be used in an immunoabsorption process, such as an ELISA, to isolate the protein or polypeptide, as is well known in the art using standard techniques. Tissue samples which can be assayed include human tissues, e.g., testis. These antibodies are also useful in diagnostic assays or as an active ingredient in a pharmaceutical composition.

The present invention also pertains to pharmaceutical compositions comprising polypeptides and other compounds described herein. For instance, a polypeptide or protein, or prodrug thereof, of the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to well known procedures, and will depend on the ultimate pharmaceutical formulation desired. Methods of introduction of exogenous polypeptides at the site of treatment include, but are not limited to, intradermal, intramuscular, intraperitoneal, intravenous, subcutaneous, oral and intranasal. Other suitable methods of introduction can also include gene therapy, rechargeable or biodegradable devices and slow release polymeric devices. The pharmaceutical compositions of this invention can also be administered as part of a combinatorial therapy with other agents.

The invention further provides kits comprising at least all or a portion of the nucleic acid molecules as described herein. Often, the kits contain one or more pairs of oligonucleotides which hybridize to a particular nucleotide sequence. In some kits, the oligonucleotides are provided immobilized to a substrate. For example, the same substrate can comprise oligonucleotide probes for detecting at least 10, 100 or more nucleic acid sequences. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

Screening Assays

The invention provides a method (also referred to herein as a “screening assay”) for identifying modulators, i.e., candidate or test compounds or agents (e.g., antisense, polypeptides, peptidomimetics, small molecules or other drugs) which bind to nucleic acid molecules, polypeptides or proteins described herein or have a stimulatory or inhibitory effect on, for example, expression or activity of the nucleic acid molecules, polypeptides or proteins of the invention.

In one embodiment, the invention provides assays for screening candidate or test compounds which bind to or modulate the activity of protein or polypeptide described herein or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. U.S.A., 90:6909; Erb et al. (1994) Proc. Natl. Acad. Sci. U.S.A., 91:11422; Zuckermann et al. (1994). J. Med. Chem., 37:2678; Cho et al. (1993) Science, 261:1303; Carell et al. (1994) Angew. Chem. Int. Ed. Engl., 33:2059; Carell et al. (1994) Angew. Chem. Int. Ed. Engl., 33:2061; and in Gallop et al. (1994) J. Med. Chem., 37:1233.

Libraries of compounds may be presented in solution (e.g., Houghten (1992)Biotechniques, 13:412-421), or on beads (Lam (1991) Nature, 354:82-84), chips (Fodor (1993) Nature, 364;555-556), bacteria (Ladner USP 5,223,409), spores (Ladner U.S. Pat. No. '409), plasmids (Cull et al.(1992) Proc. Natl. Acad. Sci. U.S.A., 89:1865-1869) or on phage (Scott and Smith (1990) Science, 249:386-390); (Devlin (1990) Science, 249:404-406); (Cwirla et al. (1990) Proc. Natl. Acad. Sci. USA, 97:6378-6382); (Felici (1991) J. Mol. Biol., 222:301-310); (Ladner supra).

In one embodiment, an assay is a cell-based assay in which a cell which expresses an encoded protein which is contacted with a test compound and the ability of the test compound to bind to the encoded protein is determined. The cell, for example, can be of mammalian origin, such as from the testis. Determining the ability of the test compound to bind to the encoded protein can be accomplished, for example, by coupling the test compound with a radioisotope or enzymatic label such that binding of the test compound to the encoded protein can be determined by detecting the labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with the encoded protein without the labeling of any of the interactants. For example, a microphysiometer can be used to detect the interaction of a test compound with the encoded protein without the labeling of either the test compound or the encoded protein. McConnell, H. M. et al. (1992) Science, 257:19061912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between encoded protein and the test compound.

In one embodiment, an assay is a cell-based assay comprising contacting a cell expressing a particular target molecule described herein with a test compound and determining the ability of the test compound to modulate or alter (e.g. stimulate or inhibit) the activity of the target molecule. Determining the ability of the test compound to modulate the activity of the target molecule can be accomplished, for example, by determining the ability of the target molecule to bind RNA.

In yet another embodiment, an assay of the present invention is a cell-free assay in which protein of the invention or biologically active portion thereof is contacted with a test compound and the ability of the test compound to bind to the protein or biologically active portion thereof is determined. Binding of the test compound to the protein can be determined either directly or indirectly as described above. In one embodiment, the assay includes contacting the protein or biologically active portion thereof with a known compound which binds the protein to form an assay mixture, contacting the assay mixture with a test compound, and determining the ability of the test compound to interact with the protein. Determining the ability of the test compound to interact with the protein comprises determining the ability of the test compound to preferentially bind to the protein or biologically active portion thereof as compared to the known compound.

In another embodiment, the assay is a cell-free assay in which a protein of the invention or biologically active portion thereof is contacted with a test compound and the ability of the test compound to modulate or alter (e.g., stimulate or inhibit) the activity of the protein or biologically active portion thereof is determined. Determining the ability of the test compound to modulate the activity of the protein can be accomplished, for example, by determining the ability of the protein to bind to a known target molecule by one of the methods described above for determining direct binding. Determining the ability of the protein to bind to a target molecule can also be accomplished using a technology such as real-time Bimolecular Interaction Analysis (BIA). Sjolander, S. and Urbaniczky, C. (1991) Anal. Chem., 63:2338-2345 and Szabo et al. (1995) Curr. Opin. Struct. Biol., 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another embodiment, the cell-free assay involves contacting a protein of the invention or biologically active portion thereof with a known compound which binds the protein to form an assay mixture, contacting the assay mixture with a test compound, and determining the ability of the test compound to interact with the protein, wherein determining the ability of the test compound to interact with the protein comprises determining the ability of the protein to preferentially bind to or modulate the activity of a target molecule.

The cell-free assays of the present invention are amenable to use of both soluble and/or membrane-bound forms of isolated proteins. In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoy-N-methylglucamide, decanoyl-N-methylglucamide, Triton® X100, Triton® X-114, Thesit®, Isotridecypoly (ethylene glycol ether)ₙ,3-[(3-cholamidopropyl) dimethylamminio]-1-propane sulfonate (CHAPS), 3-[(3-cholamidopropyl) dimethylamminio]-2-hydroxy-1-propane sulfonate (CHAPSO), or N-dodecyl-N, N-dimethyl-3-ammonio- 1 -propane sulfonate.

In more than one embodiment of the above assay methods of the present invention, it may be desirable to immobilize either the protein or its target molecule to facilitate separation of complexed from uncomplexed forms of one or both of the proteins, as well as to accommodate automation of the assay. Binding of a test compound to the protein, or interaction of the protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or protein of the invention, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, either a protein of the invention or a target molecule can be immobilized utilizing conjugation of biotin and streptavidin. Biotinylated protein of the invention or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a protein of the invention or target molecules, but which do not interfere with binding of the protein to its target molecule, can be derivatized to the wells of the plate, and unbound target or protein trapped in the wells by antibody conjugation. Methods for detecting such complexes, in addition to those described above for the GST-immobilized complexes, include immunodetection of complexes using antibodies reactive with the protein or target molecule, as well as enzyme-linked assays which rely on detecting an enzymatic activity associated with the protein or target molecule.

In another embodiment, modulators of expression of nucleic acid molecules of the invention are identified in a method wherein a cell is contacted with a candidate compound and the expression of appropriate mRNA or protein in the cell is determined. The level of expression of appropriate mRNA or protein in the presence of the candidate compound is compared to the level of expression of mRNA or protein in the absence of the candidate compound. The candidate compound can then be identified as a modulator of expression based on this comparison. For example, when expression of mRNA or protein is greater (statistically significantly greater) in the presence of the candidate compound than in its absence, the candidate compound is identified as a stimulator or enhancer of the mRNA or protein expression. Alternatively, when expression of the mRNA or protein is less (statistically significantly less) in the presence of the candidate compound than in its absence, the candidate compound is identified as an inhibitor of the mRNA or protein expression. The level of mRNA or protein expression in the cells can be determined by methods described herein for detecting mRNA or protein.

In yet another aspect of the invention, the proteins of the invention can be used as “bait proteins” in a two-hybrid assay or three-hybrid assay (see, e.g., U.S. Pat. No. 5,283,317; Zervos et al. (1993) Cell, 72:223-232; Madura et al. (1993) J. Biol. Chem., 268:12046-12054; Bartel et al. (1993) Biotechniques, 14:920-924; Iwabuchi et al. (1993) Oncogene, 8:1693-1696; and Brent WO94/10300), to identify other proteins (captured proteins) which bind to or interact with the proteins of the invention and modulate their activity. Such captured proteins are also likely to be involved in the propagation of signals by the proteins of the invention as, for example, downstream elements of a protein-mediated signaling pathway. Alternatively, such captured proteins are likely to be cell-surface molecules associated with non-protein-expressing cells, wherein such captured proteins are involved in signal transduction.

The two-hybrid system is based on the modular nature of most transcription factors, which consist of separable DNA-binding and activation domains. Briefly, the assay utilizes two different DNA constructs. In one construct, the gene that codes for a protein of the invention is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected, and cell colonies containing the functional transcription factor can be isolated and used to obtain the cloned gene which encodes the protein which interacts with the protein of the invention.

This invention further pertains to novel agents identified by the above-described screening assays. Accordingly, it is within the scope of this invention to further use an agent identified as described herein in an appropriate animal model. For example, an agent identified as described herein (e.g., a modulating agent, an antisense nucleic acid molecule, a specific antibody, or a protein-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Predictive Medicine

The present invention also pertains to the field of predictive medicine in which diagnostic assays, prognostic assays, and monitoring clinical trails are used for prognostic (predictive) purposes to thereby treat an individual prophylactically, e.g., for infertility. Accordingly, one aspect of the present invention relates to diagnostic assays for determining protein and/or nucleic acid expression as well as activity of proteins of the invention, in the context of a biological sample (e.g., blood, serum, cells, tissue) to thereby determine whether an individual is afflicted with a disease or disorder, or is at risk of developing a disorder, associated with aberrant expression or activity, such as infertility. The invention also provides for prognostic (or predictive) assays for determining whether an individual is at risk of developing a disorder associated with activity or expression of proteins or nucleic acids of the invention. For example, mutations in DAZ gene can be assayed in a biological sample.

Another aspect of the invention pertains to monitoring the influence of agents (e.g., drugs, compounds) on the expression or activity of proteins of the invention in clinical trials.

These and other agents are described in further detail in the following sections.

Diagnostic Assays

An exemplary method for detecting the presence or absence of proteins or nucleic acids of the invention in a biological sample involves obtaining a biological sample from a test subject and contacting the biological sample with a compound or an agent capable of detecting the protein, or nucleic acid (e.g., mRNA, genomic DNA) that encodes the protein, such that the presence of the protein or nucleic acid is detected in the biological sample. A preferred agent for detecting mRNA or genomic DNA is a labeled nucleic acid probe capable of hybridizing to mRNA or genomic DNA sequences described herein. The nucleic acid probe can be, for example, a full-length nucleic acid, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of SEQ ID NO: 1, 3, 5, 7, 9, 10, 11, 12, 13, 14, 15, 16, 17, 18, 19, 20, 21, 22 or 23, or a nucleic acid molecule which encodes SEQ ID NO: 2, 4 or 6, or the complement of SEQ ID NO: 1, 3, 5, 7, 9, 10, 11, 12, 13, 14, 15, 16, 17, 18, 19, 20, 21, 22 or 23, or a portion thereof. Other suitable probes for use in the diagnostic assays of the invention are described herein.

A preferred agent for detecting proteins of the invention is an antibody capable of binding to the protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, calls and biological fluids isolated from a subject, as well as tissues, cells and fluids present within a subject. That is, the detection method of the invention can be used to detect mRNA, protein, or genomic DNA of the invention in a biological sample in vitro as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled anti-protein antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample or biopsy isolated by conventional means from a subject.

In another embodiment, the methods further involve obtaining a control biological sample from a control subject, contacting the control sample with a compound or agent capable of detecting protein, mRNA, or genomic DNA of the invention, such that the presence of protein, mRNA or genomic DNA is detected in the biological sample, and comparing the presence of protein, mRNA or genomic DNA in the control sample with the presence of protein, mRNA or genomic DNA in the test sample.

In one embodiment, the present invention is a method of diagnosing reduced sperm count associated with an alteration in the gene referred to herein as the DAZ gene. Any man may be assessed with this method of diagnosis. In general, the man will have been at least preliminarily assessed, by another method, as having a reduced sperm count. By combining nucleic acid probes derived either from the isolated native sequence or cDNA sequence of the gene, or from the primers disclosed in Table 2, with the DNA from a sample to be assessed, under conditions suitable for hybridization of the probes with unaltered complementary nucleotide sequences in the sample but not with altered complementary nucleotide sequences, it can be determined whether the patient possesses the intact gene. If the gene is unaltered, it may be concluded that the alteration of the gene is not responsible for the reduced sperm count. This invention may also be used in a similar method wherein the hybridization conditions are such that the probes will hybridize only with altered DNA and not with unaltered sequences. The hybridized DNA can also be isolated and sequenced to determine the precise nature of the alteration associated with the reduced sperm count. DNA assessed by the present method can be obtained from a variety of tissues and body fluids, such as blood or semen. In one embodiment, the above methods are carried out on DNA obtained from a blood sample.

The invention also encompasses kits for detecting the presence of proteins or nucleic acid molecules of the invention in a biological sample. For example, the kit can comprise a labeled compound or agent capable of detecting protein or mRNA in a biological sample; means for determining the amount of in the sample; and means for comparing the amount of in the sample with a standard. The compound or agent can be packaged in a suitable container. The kit can further comprise instructions for using the kit to detect protein or nucleic acid.

Prognostic Assays

The diagnostic methods described herein can furthermore be utilized to identify subjects having or at risk of developing a disease or disorder associated with aberrant expression or activity of proteins and nucleic acid molecules of the invention. For example, the assays described herein, such as the preceding diagnostic assays or the following assays can be utilized to identify a subject having or at risk of developing a disorder associated with protein or nucleic acid expression or activity such as infertility. Alternatively, the prognostic assays can be utilized to identify a subject having or at risk for developing a disorder such as infertility. Thus, the present invention provides a method for identifying a disease or disorder associated with aberrant expression or activity of proteins or nucleic acid molecules of the invention, in which a test sample is obtained from a subject and protein or nucleic acid (e.g., mRNA, genomic DNA) is detected, wherein the presence of protein or nucleic acid is diagnostic for a subject having or at risk of developing a disease or disorder associated with aberrant expression or activity of the protein or nucleic acid sequence of the invention. As used herein, a “test sample” refers to a biological sample obtained from a subject of interest. For example, a test sample can be a biological fluid (e.g., serum), cell sample, or tissue (e.g., testis tissue).

Furthermore, the prognostic assays described herein can be used to determine whether a subject can be administered an agent (e.g., an agonist, antagonist, peptidomimetic, protein, polypeptide, nucleic acid, small molecule, or other drug candidate) to treat a disease or disorder associated with aberrant expression or activity of a protein or nucleic acid molecule of the invention. For example, such methods can be used to determine whether a subject can be effectively treated with an agent for a disorder, such as infertility. Alternatively, such methods can be used to determine whether a subject can be effectively treated with an agent for infertility. Thus, the present invention provides methods for determining whether a subject can be effectively treated with an agent for a disorder associated with aberrant expression or activity of a protein or nucleic acid of the present invention, in which a test sample is obtained and protein or nucleic acid expression or activity is detected (e.g., wherein the abundance of particular protein or nucleic acid expression or activity is diagnostic for a subject that can be administered the agent to treat a disorder associated with aberrant expression or activity.)

The methods of the invention can also be used to detect genetic alterations in genes or nucleic acid molecules of the present invention, thereby determining if a subject with the altered gene is at risk for a disorder characterized by aberrant development, aberrant cellular differentiation, aberrant cellular proliferation or an aberrant hematopoietic response. In preferred embodiments, the methods include detecting, in a sample of cells from the subject, the presence or absence of a genetic alteration characterized by at least one of an alteration affecting the integrity of a gene encoding a particular protein, or the mis-expression of the gene. For example, such genetic alterations can be detected by ascertaining the existence of at least one of (1) a deletion of one or more nucleotides; (2) an addition of one or more nucleotides; (3) a substitution of one or more nucleotides, (4) a chromosomal rearrangement; (5) an alteration in the level of a messenger RNA transcript; (6) aberrant modification, such as of the methylation pattern of the genomic DNA; (7) the presence of a non-wild type splicing pattern of a messenger RNA transcript; (8) a non-wild type level; (9) allelic loss; and (10) inappropriate post-translational modification. As described herein, there are a large number of assay techniques known in the art which can be used for detecting alterations in a particular gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such an anchor PCR or RACE PCR, or, alternatively, in a ligation chain reaction (LCR) (see, e.g., Landegran et al. (1988) Science, 241:1077-1080; and Nakazawa et al. (1994) PNAS, 91:360-364), the latter of which can be particularly useful for detecting point mutations (see Abravaya et al. (1995) Nucleic Acids Res., 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA, 87:1874-1878), transcriptional amplification system (Kwoh, D. Y. et al., (1989) Proc. Natl. Acad. Sci. USA, 86:11731177), Q-Beta Replicase (Lizardi, P. M. et al.,(1988) Bio/Technology, 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a given gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for sample, U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotide probes (Cronin, M. T. et al. (1996) Human Mutation, 7:244-255; Kozal, M. J. et al. (1996) Nature Medicine, 2:753-759). For example, genetic mutations can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the gene and detect mutations by comparing the sequence of the gene from the sample with the corresponding wild-type (control) gene sequence. Examples of sequencing reactions include those based on techniques developed by Maxim and Gilbert ((1997) PNAS, 74:560) or Sanger ((1977) PNAS, 74:5463). It is also contemplated that any of a variety of automated sequencing procedures can be utilized when performing the diagnostic assays ((1995) Biotechniques, 19:448), including sequencing by mass spectrometry (see, e.g., PCT International Publication No. WO 94/16101; Cohen et al. (1996) Adv. Chromatogr., 36:127-162; and Griffin et al. (1993) Appl. Biochem. Biotechnol., 38:147-159).

Other methods for detecting mutations include methods in which protection from cleavage agents is used to detect mismatched bases in RNA/RNA or RNA/DNA heteroduplexes (Myers et al. (1985) Science, 230:1242). In general, the art technique of “mismatch cleavage” starts by providing heteroduplexes of formed by hybridizing (labeled) RNA or DNA containing the wild-type sequence with potentially mutant RNA or DNA obtained from a tissue sample. The double-standard duplexes are treated with an agent which cleaves single-stranded regions of the duplex such as which will exist due to base pair mismatches between the control and sample strands. For instance, RNA/DNA duplexes can be treated with Rnase and DNA/DNA hybrids treated with SI nuclease to enzymatically digest the mismatched regions. After digestion of the mismatched regions, the resulting material is then separated by size on denaturing polyacrylamide gels to determine the site of mutation. See, for example Cotton et al (1988) Proc. Natl. Acad. Sci. USA, 85:4397; Saleeba et al. (1992) Methods Enzymol., 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis, 15:1657-1662). According to an exemplary embodiment, a probe based on an nucleotide sequence of the invention is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA, 86:2766, see also Cotton (1993) Mutat Res, 285:125-144; and Hayashi (1992) Genet Anal. Tech. Appl., 9:73-79). Single-stranded DNA fragments of sample and control nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet., 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature, 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem., 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature, 324:163); Saiki et al. (1989) Proc. Natl. Acad. Sci. USA, 86:6320). Such allele-specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res., 17:2437-2448) or at the extreme 3′ end of one primer where, under appropriate conditions, mismatch can prevent, or reduce polymerase extension (Prossner (1993) Tibtech, 11:238). In addition it may be desirable to introduce a novel restriction site in the region of the mutation to create cleavage-based detection (Gasparini et al. (1992) Mol. Cell Probes, 6:1). It is anticipated that in certain embodiments amplification may also be performed using Taq ligase for amplification (Barany (1991) Proc. Natl. Acad. Sci. USA, 88:189). In such cases, ligation will occur only if there is a perfect match at the 3′end of the 5′sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnose patients exhibiting symptoms or family history of a disease or illness involving a gene of the present invention. Any cell type or tissue in which the gene is expressed may be utilized in the prognostic assays described herein.

Monitoring of Effects During Clinical Trials

Monitoring the influence of agents (e.g., drugs, compounds) on the expression or activity of nucleic acid molecules or proteins of the present invention (e.g., modulation of cellular signal transduction, regulation of gene transcription in a cell involved in development or differentiation, regulation of cellular proliferation) can be applied not only in basic drug screening, but also in clinical trials. For example, the effectiveness of an agent determined by a screening assay as described herein to increase gene expression, protein levels, or up-regulate protein activity, can be monitored in clinical trails of subjects exhibiting decreased gene expression, protein levels, or down-regulated protein activity. Alternatively, the effectiveness of an agent determined by a screening assay to decrease gene expression, protein levels, or down-regulate protein activity, can be monitored in clinical trials of subjects exhibiting increased gene expression, protein levels, or up-regulated protein activity. In such clinical trials, the expression or activity of the specified gene and, preferably, other genes that have been implicated in, for example, a proliferative or infertility disorder can be used as a “read out” or markers of the phenotype of a particular cell.

For example, and not by way of limitation, genes that are modulated in cells by treatment with an agent (e.g., compound, drug or small molecule) which modulates protein activity (e.g., identified in a screening assay as described herein) can be identified. Thus, to study the effect of agents on infertility, proliferative disorders, developmental or differentiative disorder, or hematopoietic disorder, for example, in a clinical trial, cells can be isolated and RNA prepared and analyzed for the levels of expression of the specified gene and other genes implicated in the infertility, proliferative disorder, developmental or differentiative disorder, or hematopoietic disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of the specified gene or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, polypeptide, nucleic acid, small molecule, or other drug candidate identified by the screening assays described herein) comprising the steps of (I) obtaining a pre-administration sample from a subject prior to administration of the agent; (ii) detecting the level of expression of a specified protein, mRNA, or genomic DNA of the invention in the pre-administration sample; (iii) obtaining one or more post-administration samples from the subject; (iv) detecting the level of expression or activity of the protein, mRNA, or genomic DNA in the post-administration samples; (v) comparing the level of expression or activity of the protein, mRNA, or genomic DNA in the pre-administration sample with the protein, mRNA, or genomic DNA in the post-administration sample or samples; and (vi) altering the administration of the agent to the subject accordingly. For example, increased administration of the agent may be desirable to increase the expression or activity of the protein or nucleic acid molecule to higher levels than detected, i.e., to increase effectiveness of the agent. Alternatively, decreased administration of the agent may be desirable to decrease effectiveness of the agent. According to such an embodiment, protein or nucleic acid expression or activity may be used as an indicator of the effectiveness of an agent, even in the absence of an observable phenotypic response.

Methods of Treatment

The present invention provides for both prophylactic and therapeutic methods of treating a subject at risk of (or susceptible to) a disorder or having a disorder associated with aberrant expression or activity of proteins or nucleic acids of the invention. With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with the molecules of the present invention or modulators according to that individual's drug response genotype. Pharmacogenomics allows a clinician or physician to target prophylactic or therapeutic treatments to patients who will most benefit from the treatment and to avoid treatment of patients who will experience toxic drug related side effects.

This invention has utility in methods of treating disorders of reduced sperm count associated with alteration of the DAZ gene or a member of the DAZ gene family. These genes may be used in a method of gene therapy, whereby the gene or a gene portion encoding a functional protein is inserted into cells in which the functional protein is expressed and from which it is generally secreted to remedy the deficiency caused by the defect in the native gene.

The invention described herein also has application to the area of male contraceptives, since alteration of the DAZ gene produces the functional effects which are desirable in a male contraceptive, e.g., failure to produce sperm without other apparent physiological consequences. Thus, the present invention also relates to agents or drugs, such as, but not limited to, peptides or small organic molecules which mimic the activity of the altered DAZ gene product. Alternatively, the agent or drug is one which blocks or inhibits the activity or function of the unaltered DAZ gene (e.g., an oligonucleotide or a peptide). The ideal agent must enter the cell, in which it will block or inhibit the function of the DAZ gene, directly or indirectly.

Prophylactic Methods

In one aspect, the invention provides a method for preventing in a subject, a disease or condition associated with aberrant expression or activity of genes or proteins of the present invention, by administering to the subject an agent which modulates expression or at least one activity of a gene or protein of the invention. Subjects at risk for a disease which is caused or contributed to by aberrant gene expression or protein activity can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein. The prophylactic methods of the present invention are further discussed in the following subsections.

Therapeutic Methods

Another aspect of the invention pertains to methods of modulating expression or activity of genes or proteins of the invention for therapeutic purposes. The modulatory method of the invention involves contacting a cell with an agent that modulates one or more of the activities of the specified protein associated with the cell. An agent that modulates protein activity can be an agent as described herein, such as a nucleic acid or a protein, a naturally-occurring target molecule of a protein described herein, a polypeptide, a peptidomimetic, or other small molecule. In one embodiment, the agent stimulates one or more protein activities. Examples of such stimulatory agents include active protein as well as a nucleic acid molecule encoding the protein that has been introduced into the cell. In another embodiment, the agent inhibits one or more protein activities. Examples of such inhibitory agents include antisense nucleic acid molecules and anti-protein antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a protein or nucleic acid molecule of the invention. In one embodiment, the method involves administering an agent (e.g., an agent identified by a screening assay described herein), or combination of agents that modulates (e.g., up-regulates or down-regulates) expression or activity of a gene or protein of the invention. In another embodiment, the method involves administering a protein or nucleic acid molecule of the invention as therapy to compensate for reduced or aberrant expression or activity of the protein or nucleic acid molecule.

Stimulation of protein activity is desirable in situations in which the protein is abnormally down-regulated and/or in which increased protein activity is likely to have a beneficial effect. Likewise, inhibition of protein activity is desirable in situations in which the protein is abnormally up-regulated and/or in which decreased protein activity is likely to have a beneficial effect. One example of such a situation is where a subject has a disorder characterized by aberrant development or cellular differentiation, for example infertility. Another example of such a situation is where the subject has a proliferative disease (e.g., cancer) or a disorder characterized by an aberrant hematopoietic response.

Pharmacogenomics

The molecules of the present invention, as well as agents, or modulators which have a stimulatory or inhibitory effect on the protein activity (e.g., gene expression) as identified by a screening assay described herein can be administered to individuals to treat (prophylactically or therapeutically) disorders (e.g., proliferative or developmental disorders) associated with aberrant protein activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a molecule of the invention or modulator thereof, as well as tailoring the dosage and/or therapeutic regimen of treatment with such a molecule or modulator.

Pharmacogenomics deals with clinically significant hereditary variations in the response to drugs due to altered drug disposition and abnormal action in affected persons. See e.g., Eichelbaum, M., Clin Exp Pharmacol. Physiol., (1996) 23(10-11):983-985 and Linder, M. W., Clin. Chem. (1997) 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1,000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a protein or a receptor of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2(NAT 2) and cytochrme P450 enzymes CYP2D6 and CYP2C19) has provided an explanation as to why some patients do not obtain the expected drug effects or show exaggerated drug response and serious toxicity afer taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a molecule or modulator of the present invention) can given an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a molecule or modulator of the invention, such as a modulator identified by one of the exemplary screening assays described herein.

The present invention is also drawn to a method for distinguishing a DAZ gene of interest from other DAZ genes by detecting sequence family variants. The method comprises conducting at least one amplification reaction to amplify at least one region of a DAZ gene; digesting the amplified product with a restriction endonuclease; and detecting products of the digestion, wherein the products of the digestion distinguishes the DAZ gene of interest from other DAZ genes.

The invention will be further exemplified by the following non-limiting examples. The teachings of all references, patents, patent applications and websites cited herein are incorporated herein by reference in their entirety.

### EXEMPLIFICATION

Chromosomal Organization of DAZ Genes

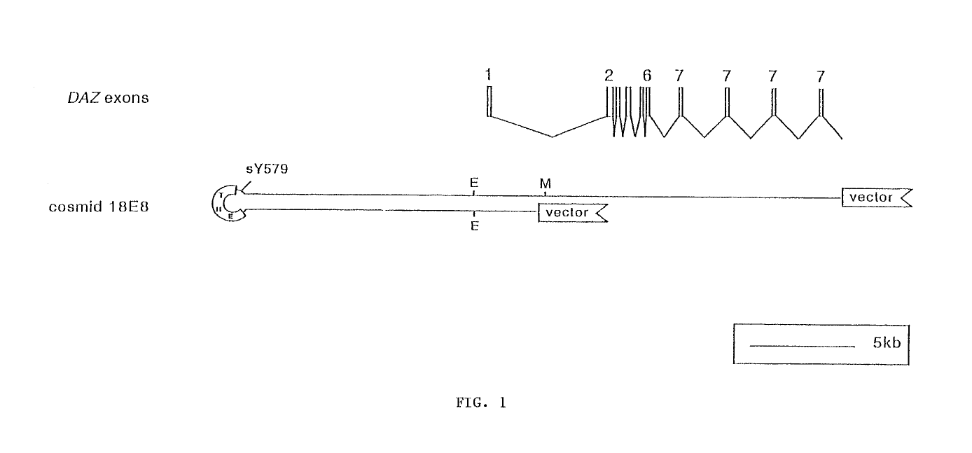

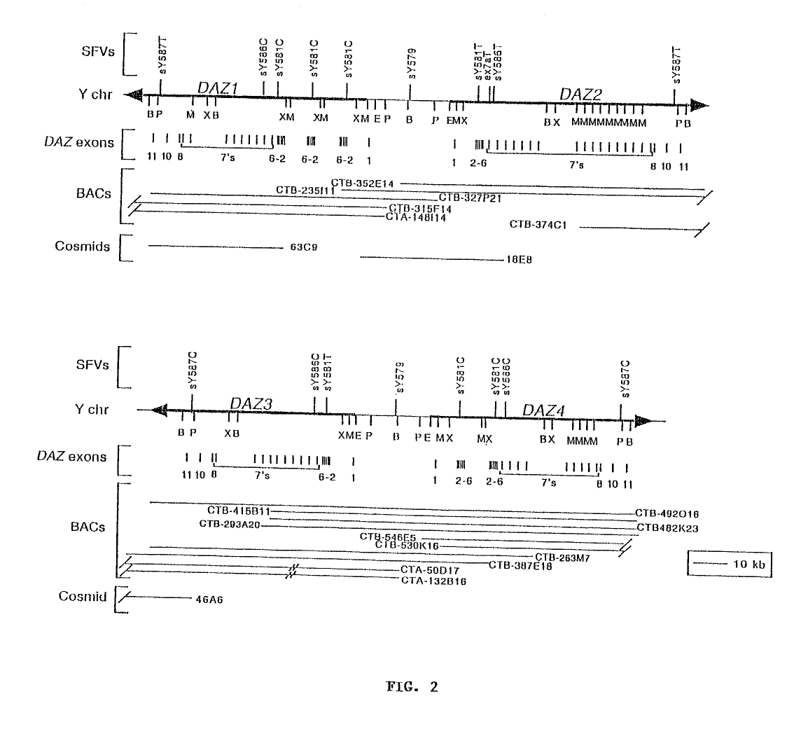

Sequencing and mapping of cosmid 18E8 revealed a nearly perfect inverted duplication comprising most of the cosmid's insert (FIG. 1). FIG. 1 shows a schematic diagram of the inverted duplication in cosmid 18E8. DAZ exons are shown above and a small nonduplicated segment containing a 1.9-kb THE element and STS marker sY579 is located between the arms of the duplication. Eagi (E) and MluI (M) restriction sites are also shown. One arm of the inverted sequence contains DAZ exons 1 through 7d. The other arm, which extends to the cosmid's cloning site, contains a second copy of exon 1 (and part of intron 1) in reverse orientation. A non-duplicated segment of 2.1 kb (including a THE element) lies between the inverted repeats.

The sequencing of cosmid 18E8 suggested that at least one inverted pair of DAZ genes might exist on the Y chromosomes. Fluorescence in situ hybridization (FISH) analysis confirmed this hypothesis DAZ cosmid probes were hybridized to human male chromatin in three different states of condensation: 1) in interphase fibroblast nuclei, 2) in extended chromatin fibers from spermatozoa and 3) in fully extended chromatin fibers from lymphocytes prepared as described above. In all three cases the experiment was repeated with samples from multiple, unrelated men.

Fluorescence in situ Hybridization (FISH)

One or two-color FISH was performed according to standard procedures (Redeker, 1994). Probes were labeled with biotin or digoxigenin, hybridized to target DNA, and detected by avidin or anti-digoxigenin antibodies conjugated to fluorocliromes Cy3 or fluorescein.

Extended chromatin fibers from spermatozoa were prepared as described previously (Haaf and Ward, 1995) with minor modifications. Sperm were isolated by density centrifugation on 70% Percoll gradient, washed twice in phosphate-buffered saline (PBS), resuspended in a 3:1 mixture of methanol/acetic acid to 10⁷ sperm/ml, allowed to fix for 1 h at −20° C., and dropped onto glass slides. After blow drying, slides were incubated in extraction solution (0.125% SDS, 0.2 M NaOH) for 5 min at 30° C. The solution was removed, and new extraction solution was pipetted onto one end of the slide and smeared out using a coverslip. This procedure was repeated using fixative (3:1 methanol/acetic acid). The slides were dehydrated and kept at room temperature prior to hybridization.

Extended chromatin fibers from lymphocytes were prepared using SDS/EDTA extraction (Fidlerova, 1994).

Hybridization of DAZ cosmid 18E8 (5′ DAZ) to interphase fibroblast nuclei generated two signals in 75% of nuclei examined. In remaining nuclei (25%), one signal was observed, likely from the superimposition of the two signals. By contrast, 3′ DAZ cosmid 46A6 produced four signals in 41% of nuclei examined, with the remaining nuclei exhibiting three (28%), two (24%), or one signal (7%). Superimposition of signals may account for the nuclei exhibiting three or fewer signals. These findings suggested 1) that there are four DAZ genes on the Y chromosome and 2) that the 5′ ends of the DAZ genes (two FISH signals) are in closer proximity than their 3′ ends (up to four FISH signals), consistent with head-to-head DAZ gene duplication (3←5′::5′→3′).

To achieve higher resolution, DAZ cosmids were hybridized to extended chromatin fibers from spermatozoa. There, two-color FISH with DAZ cosmids 63C9 and 46A6 revealed two large signal clusters. Within each cluster, the 46A6 signal (3′ DAZ) overlapped the outer ends of the 63C9 signal (central portion of DAZ), as expected if two head-to-head DAZ genes are present in each cluster. These studies were repeated on six other unrelated men, in each case the same pattern or two clusters was observed, with evidence of 3←5′::5′→3′ orientation within each cluster.

To examine the orientation of DAZ genes within a cluster in detail, two-color FISH on extended chromatin fibers from lymphocytes of two unrelated men was preformed. Each cluster was consistently observed to contain two DAZ genes in head-to-head orientation.

Taken together the FISH studies suggested that human Y chromosomes carry two DAZ clusters, each containing two DAZ genes in 3′←5′::5′→3′ orientation.

DAZ BACs were isolated from human male genomic libraries prepared at the California Institute of Technology (Shizuya, 1992). High-density library filters (Research Genetics) were probed using radiolabeled PCR products corresponding to DAZ STSs. A total of 16 DAZ BACs were identified. Three BACs (prefixed with CTA) derive from DNA of one male donor. The remaining 13 BACs (prefixed with CTB) derive from a second, unrelated male donor. BAC DNA was isolated using alkaline lysis and column chromatography (Qiagen) using pre-heated elution buffer.

DAZ probes were used to screen human male BAC libraries providing an estimated 4-to-5-fold coverage of the Y chromosome 16 DAZ BAC clones were identified and characterized. A physical map of the four DAZ genes based on studies of these BAC clones is presented in FIG. 2. FIG. 2 shows genomic organization of four DAZ genes in two clusters as inferred from analysis of BAC and cosmid clones. Oversized arrows indicate direction of trascription of DAZ genes. The restriction sites are: B (BamHI), P (PmeI), M (MluI), X (XhoI) and E (EagI). Shown above the arrows are sequence family variants (SFVS; FIG. 7) that distinguish between DAZ genes; e.g., “sY586C” indicates that a C is present at the variable nucleotide position in sY586. The position of STS sY586 is also shown. Below the arrows, DAZ exons are numbered. BACs prefixed with CTA are from a different male donor than BACs prefixed with CTB. The small slashes on the DAZ3-containing CTA BACs (CTA-50D17 and CTA-132B 16) denot that they contain two more 2.4-kb repeats (two more copies of exon 7) than the DAZ3-containing CTA BACs.

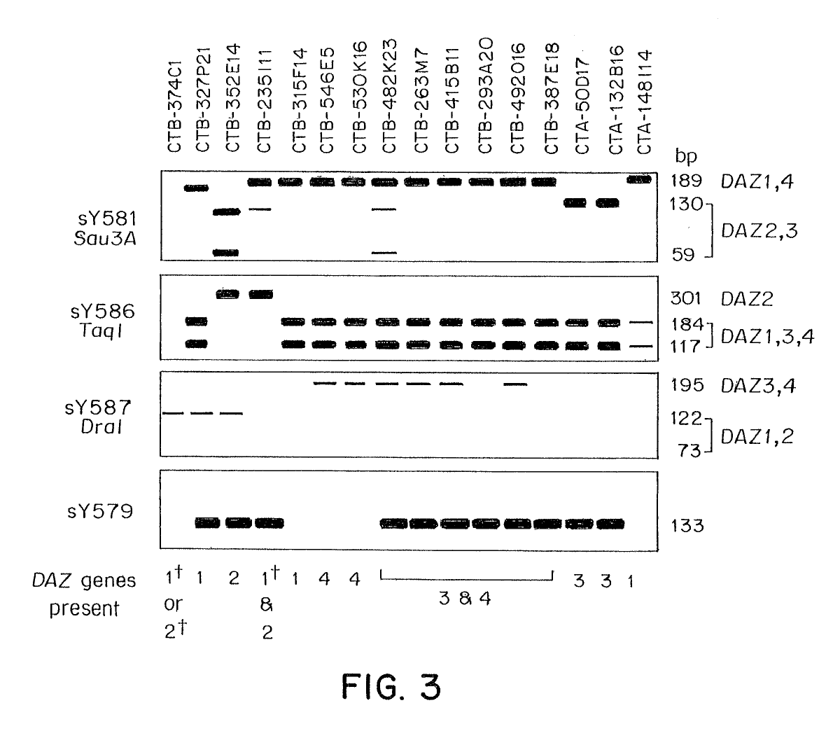

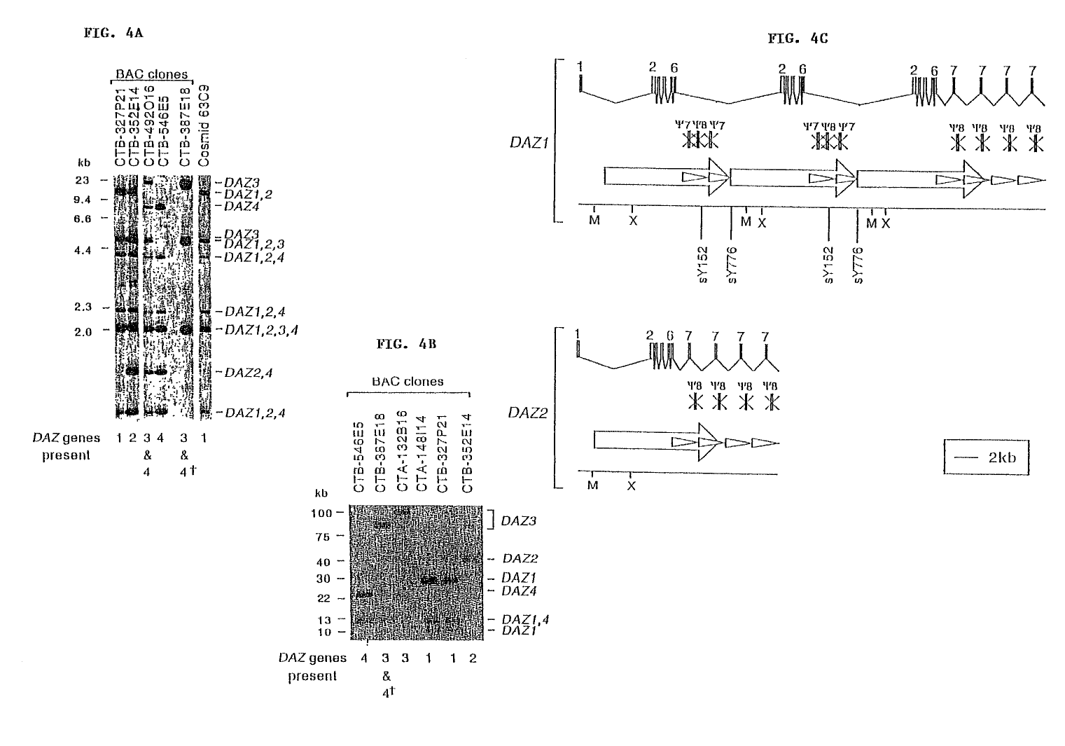

PCR/restriction-digest assays were developed to type the BACs for single nucleotide variants. Typing of the 16 BACs for three sequence variants (sY581/Sau3A, sY586/Taq1, and sY587/Dral) revealed four distinct DAZ gene signatures—DAZ1, DAZ2, DAZ3 and DAZ4 (see FIG. 7 and FIG. 3 for details). FIG. 3 shows a gel analysis of SFVs in DAZ BAC clones scored by PCR-restriction digest analysis. In FIG. 3, the assays listed along the left are described in FIG. 7; the positions of SFVs within DAZ genes are shown in FIG. 3. The fragment sizes (in bp) and the DAZ genes giving rise to each fragment are listed along the right in FIG. 3. sY579 maps between 5′ ends of inverted DAZ genes (FIG. 1). Listed at the bottom of each lane is the DAZ gene(s) present in that BAC clone; t denotes that only a portion of the indicatd DAZ gene is present in that BAC. Nine of the 16 BACs exhibited a single signature—either DAZ1, DAZ2, DAZ3 or DAZ4— consistent with each carrying a single DAZ gene.

The seven other BACs exhibited two signatures each-either DAZ1 plus DAZ2, or DAZ3 plus DAZ4. Each of the two-signature BACs contained sY579, an STS located between the 5′ ends of the inverted DAZ genes found in cosmid 18E8 (FIG. 1). Similarly, restriction digestion and pulsed-field gel electrophoresis of these seven BACs revealed that each contained an EagI fragment of 20 kb, as also seen in 5′ cosmid 18E8 (FIG. 1). The apparent pairing of DAZ1 with DAZ2 (in BAC CTB-235111), and of DAZ3 with DAZ4 (in six independent BAC clones), suggested the precise composition of the two DAZ clusters visualized by FISH, two clusters, each containing an inverted pair of DAZ genes. DAZ1 or DAZ2 were never seen in the same BAC clone as DAZ3 or DAZ4, consistent with the DAZ1/2 and DAZ3/4 clusters being too far apart for both clusters to be captured within a BAC insert.

Family Variants (SFVS) that Distinguish Between DAZ Genes.

Three DAZ cosmids were used in these studies (Saxena, 1996). Cosmid 18E8 has an insert of 42,791 bp, corresponding to nucleotides 670 through 43,460 of BAC RP11-29003 (Genbank AC010089). As shown in FIG. 1, cosmid 18E8 encompasses the 5″ portions of two neighboring DAZ genes. Cosmid 63C9 (Genbank AC000021) contains exons 2 through 11 and thus almost an entire DAZ gene. Cosmid 46A6 (Genbank AC000022) derives from the 3′ portion of DAZ; it contains exons 8 through 111 as well as 35 kb downstream of the gene. PCR amplification was preformed in 20 μl volumes of 1.5 mM MgCl₂, 5 mM NH₄Cl, 10 mM Tris (pH 8.3), 50 mM KCI, 100 μM dNTPs, with 1 U Taq DNA polymerase and 1 μM of each primer. PCR primers and conditions are deposited in Genbank: sY581, Genbank G63906; sY586, G63907; sY587, G63908; sY579, G63909; sT776, G63910. To detect SFVs at sY581, sY586 and sY587, PCR products were digested with restriction enzymes as listed in FIG. 7.

Tandem Amplification of 10.8-kb Unit Within DAZ1and DAZ4.

Pulsed-Field Gel Electrophoresis.

DAZ BACs were sized by pulsed-field gel electrophoresis in 1% agarose using a Bio-rad CHEF-DRII system. Electrophoresis was performed for 26 h at 15° C. and 179 V with ramped switch times of 5 to 20 s. Estimated BAC sizes (including vector sequences) were as follows: CTA-50D17, 240 kb; CTA-132B16, 122 kb; CTA-148114, 110 kb; CTB-235111, 165 kb; CTB-236M7, 130 kb; CTB-293A20, 170 kb; CTB-315F14, 140 kb; CTB-327P21, 130 kb; CTB-352E14, 200 kb; CTB-374C1, 100 kb; CTB-387E18, 138 kb; CTB-415B11, 160 kb; CTB-482K23 175 kb; CTB-492016, 200 kb; CTB-530K16, 150 kb; and CTB-546E5, 135 kb.

For Southern analysis of DAZ genes, restriction-digested BACs were subjected to electrophoresis for 11 h at 14° C. and 200 V with ramped switch times of 1 to 6 s. This separated restriction fragments ranging in size from 5 to 75 kb.

Southern Blotting.

Following agarose gel electrophoresis, restriction-digested BAC and cosmid DNAs were transferred onto Genescreen Plus (NEN) membranes and hybridized with radiolabled DAZ PCR products or plasmid insert (pDP1646; 2.4-kb insert from DAZ genomic locus). Probes were labeled with ³²P-dCTP by random priming. Hybridization was carried out at 65° C. in 0.5 M NaPO₄ (pH7.5), 7% SDS. Membranes were subsequently washed at 65° C. in 0.1× SSC, 0.1% SDS three times for 20 minutes each.

The four DAZ genes were compared at a structural level by additional restriction mapping of their respective BACs. Conventional and pulsed-field Southern blotting of BAC DNAs enabled the identification of restriction fragments of particular interest. Hybridization probes employed in these studies included PCR products and synthetic oligonucleotides corresponding to specific exons, as well as plasmid sub-clones of portions of the genes. The resulting maps and inferred arrangements of exons are summarized in FIG. 2, where, in the interest of clarity, only selected restriction sites are shown.

This restriction mapping/Southern blot analysis of DAZ BACs yielded several insights. First, the four DAZ genes differ in size, as revealed most directly by pulsed-field gels following digestion with Pmel, which cuts near the 5′ and 3′ ends of all four genes. The approximate sizes of the genes are as follows: DAZ1, 65 kb; DAZ2, 70 kb; DAZ3, 50 kb and DAZ4, 55 kb.

The analysis of DAZ BACs also revealed that, in the central portions of all four genes, there are tandem arrays of a previously identified 2.4-kb unit. Previous sequencing of DAZ1 cosmid 63C9 (Saxena, 1996) had identified this genomic repeat and revealed that it contains a 72-bp exon (exon 7) encoding a 24-amino acid segment that is tandemly amplified within predicted DAZ proteins (Reijo, 1995; Yen, 1997). As shown in FIG. 4, hybridization of a 2.4-kb-repeat probe to restriction-digested BAC DNAs revealed a set of large fragments—similar to those seen in cosmid 63C9— in each of th four genes. FIG. 4A shows a Southern blot of a 2.4-kb repeat probe pDP1649 to TaqI-digested DAZ BAC and cosmid DNAs. Listed at the botton of each lane is the DAZ gene present in that BAC or cosmid clone; † denotes that only a portion of the indicated DAZ gene is present. Like cosmid 63C9, which has eight tandem 2.4-kb repeats interrupted by a LINE element (Saxena et al., 1996), all DAZ-containing BACs display multiple large hybridizing fragments. FIG. 4B shows a Southern blot of a PCR fragment spanning DAZ exons 2 and 3 to MluI-digested DAZ BAC DNAs. FIG. 4C is a schematic diagram of 5′ portions of DAZ1 and DAZ2 genes with three tandem copies or one copy, respectively of the 10.8-kb repeat (large open arrow). These and other Southern blot analyses of DAZ BACs indicated that the 2.4-kb unit is tandemly amplified in all four genes. 2.4-kb repeats shown as smaller open arrowheads. Exons and pseudoexons (Ψ) are indicated above the repeats; restriction sites M, X and positions of STS markers sY152 and sY776 (ehich detects the junction between tandem 10.8-kb repeats) are shown below. As summarized in FIG. 2, all four DAZ genes appear to contain many copies of exon 7.

Finally, our analysis of DAZ BAC clones revealed a second tandemly amplified segment within DAZ genes: a 10.8-kb unit that is triplicated in DAZ1 and duplicated in DAZ4, as summarized in FIG. 4. Nucleotide sequence analysis of DAZ cosmids previously revealed only two Mlul restriction sites within a composite DAZ transcription unit-one site in intron 1 and another site in one copy of exon 7 (Saxena, 1996). A genomic probe encompassing exons 2 and 3 was hybridized to pulse-field Southern blots of Mlul-digested BAC DNAs. As shown in FIG. 4B, a single hybridizing fragment in BACs containing either DAZ2 (BAC CTB-352E14) or DAZ3 (BAC CTA-132B16) was observed. However, three or two hybridizing Mlul fragments were observed in BACs containing DAZ1 (BACs CTA-148114 and CTB-327P21) or DAZ4 (BAC CTB546E5), respectively. These results suggested that DAZ1 and DAZ4 contained, respectively, three and two copies of exons 2-3. Additional Southern-blot studies of BAC DNAs revealed that exons 4-6 are also present three times in DAZ2 and twice in DAZ4.

cDNA Cloning and Sequencing

The single nucleotide variants used to distinguish among the DAZ genes were all located in introns. Having identified BACs corresponding to each of the four DAZ genes, the genes' coding regions were compared at the nucleotide level. For each of the four genes, exons 1 through 7a (the 5′-most copy of exon 7; Saxena, 1996) and exons 8 through 11 were sequenced, using BACs as sources of sequencing templates. As judged by this limited genomic sequence analysis, the coding regions of all four genes appeared to be intact, with no evidence of frameshift or nonsense mutations in DAZ1, DAZ2, DAZ3 or DAZ4. Indeed, only one coding sequence difference was observed among the DAZ genes: a silent C-to-T transition in exon 7a in DAZ2.

DAZ cDNA clones were identified by screening a library (HL1161X, Clontech) prepared from testes of four men; the screening methods were described previously (Reijo, 1995). Lambda phage cDNA clones were converted into pDR plasmids (pDP1575, pDP1576, pDP1678, pDP1679), or their inserts were PCR amplified and subcloned into pBluescript plasmids (pDP1680 and pDP1681, with overlapping inserts together representing a single isolate from the cDNA library).

Because of lengthy tandem repeats, DAZ cDNA clones were not amenable to nucleotide sequencing by conventional methods. Instead, sequencing was conducted from transposon inserted into cDNA subclones (Devine, 1997). Briefly, for cDNA clones pDP1575, pDP1678, pDP1679 and pDP1680, a library of recombinant plasmids carrying transposon insertions were prepared using a Primer Island Transposition Kit (PE Appliled Biosystems) in vitro. The transposition reaction was terminated by adding freshly prepared stop buffer (0.25 M EDTA, 1% SDS, 5 mg/ml proteinase K) and incubating at 65° C. for 30 minutes. Excess reagents were removed by precipitating the products with isopropanol solution (25 μl water, 25 ∥l 7.5 M ammonium acetate, 75 μl isopropanol) and washing with 70% ethanol.

The resulting plasmid DNAs were electroporated (Gene Pulser; Bio-rad) into DH10B E. Coli cells (Life Technology) at a setting of 25 μF, 200 ohm, 2.5 V. Subsequent sample preparation and DNA sequencing were carried out as described (Chen, 1996), employing primers PIP (3′-CAGGACATTGGATGCTGAGAATTCG-5′; SEQ ID NO: 24) and PIM (3′-CAGGAGCCGTCTATCCTGCTTGC-5′; SEQ ID NO: 25) with BigDye (PE Applied Biosystems) terminator chemistry. Sequence data were assembled using Phred/Phrap and edited using Consed (http://www.phrap.org).

As described herein, analysis of DAZ genomic sequences suggested that the coding sequences of all four DAZ genes were intact. However, genomic sequencing alone could not reveal whether each of the four genes was transcribed in vivo. A variety of DAZ cDNA clones were sequenced and assigned to individual DAZ genes.

17 DAZ cDNA clones from a human testes cDNA library made from RNAs pooled from four individuals. The five longest clones were selected and sequenced in their entirety. Sequencing of DAZ cDNA clones is difficult because of lengthy tandem repeats within the coding regions, and few if any DAZ cDNA clones had been fully and accurately sequenced in previous studies (see discussion in Yen, 1997). To circumvent these difficulties, transposons were inserted into the cDNA clones, thereby introducing unique priming sites for sequencing. Three of the five sequenced cDNA clones appeared to be full-length, containing a complete, intact DAZ open reading frame. By comparing cDNA and genomic sequences, the first of the full-length cDNA clones was assigned to DAZ3, the second to DAZ2, and the third to DAZ4 or DAZ1.

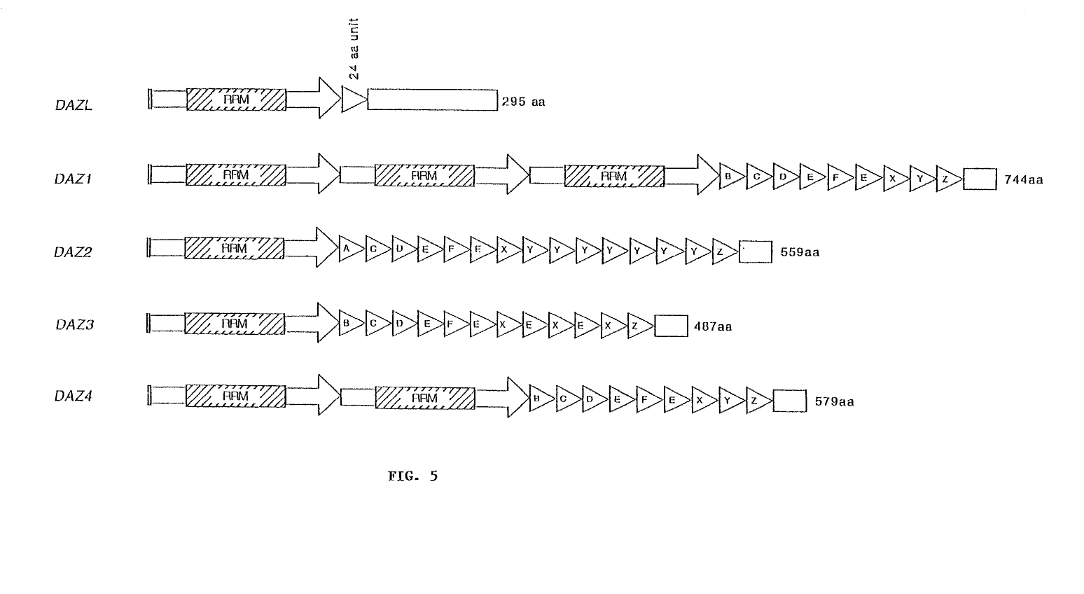

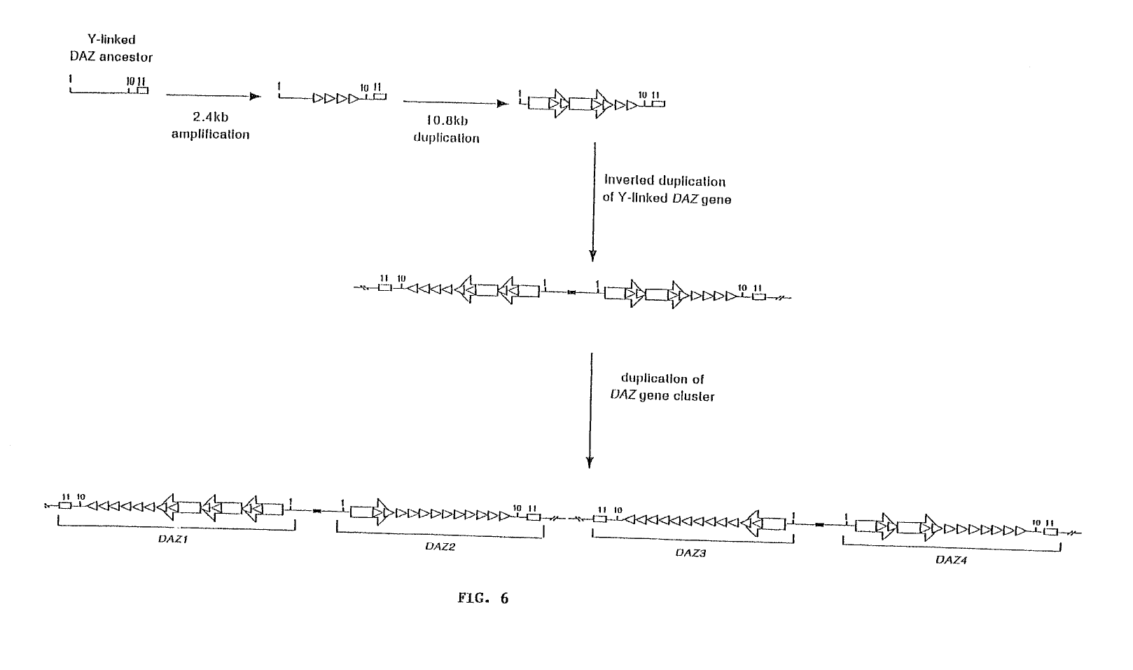

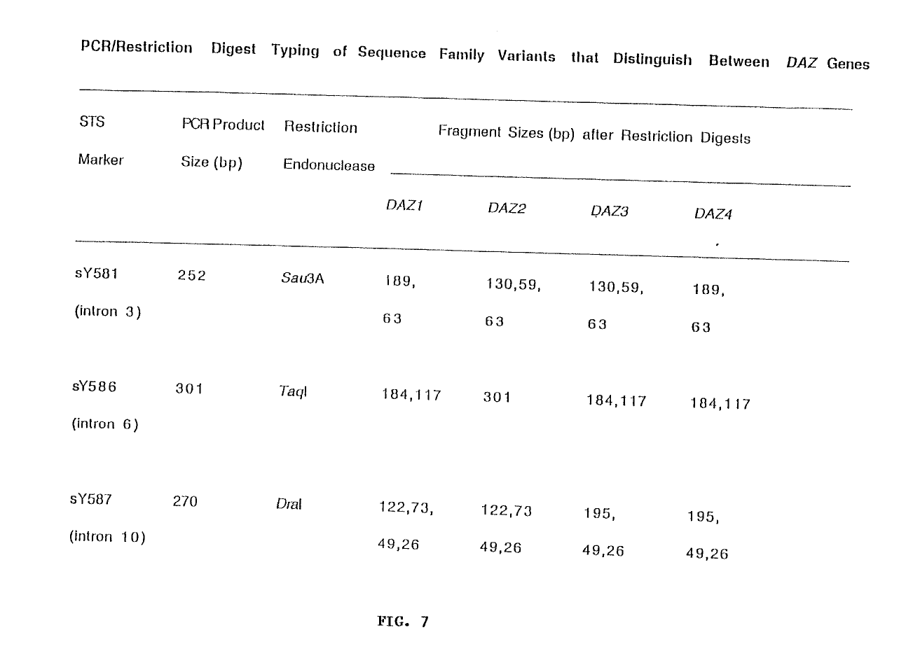

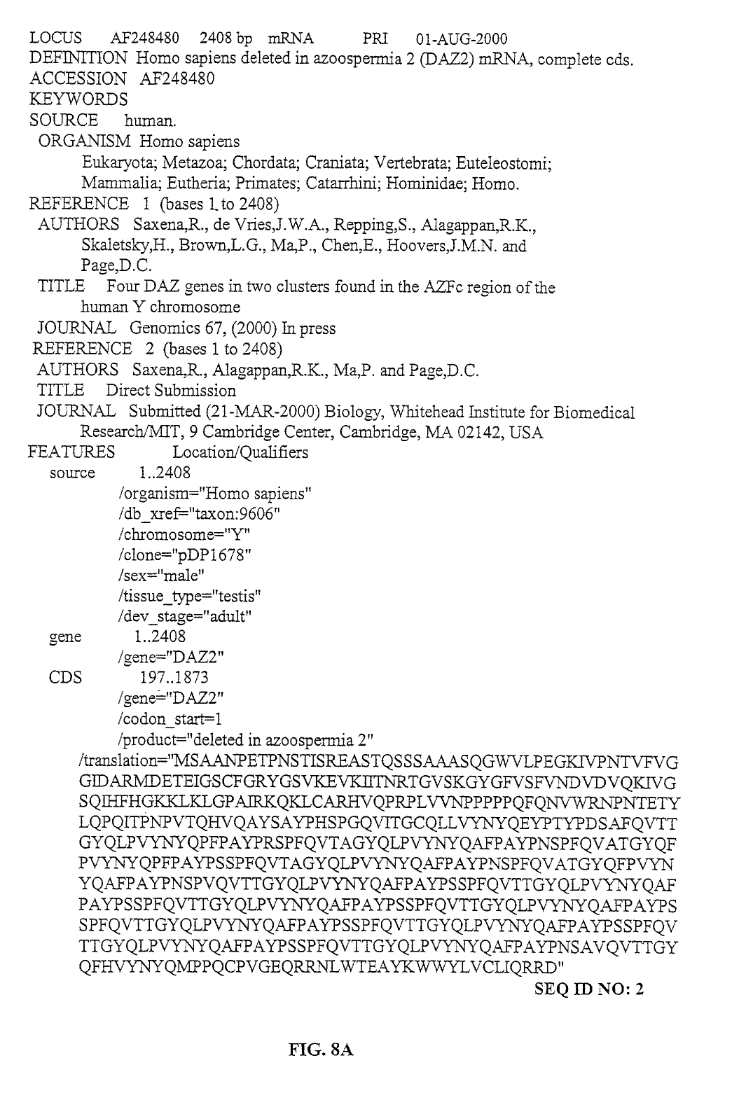

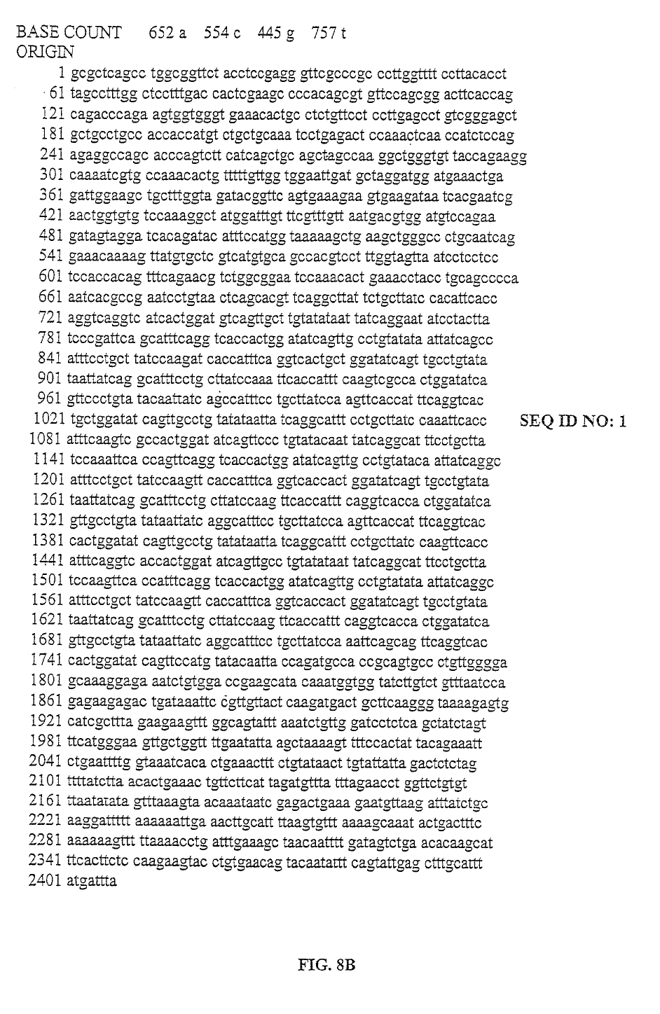

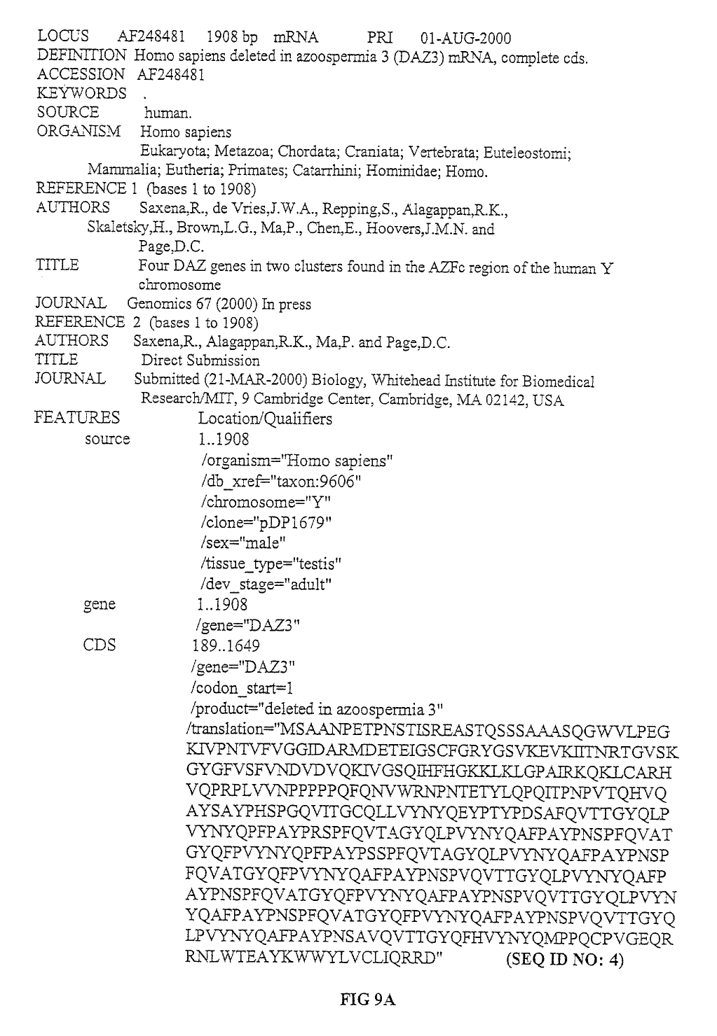

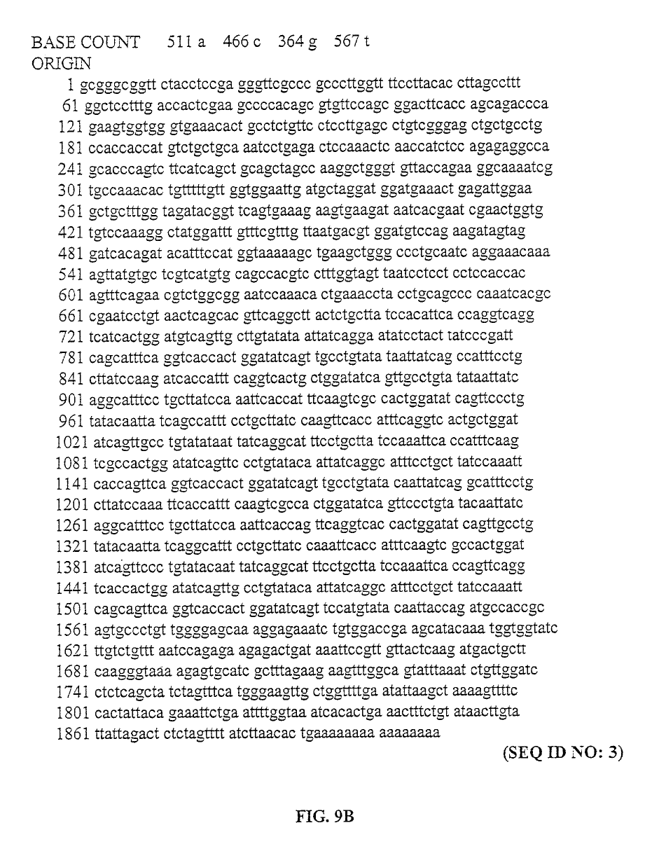

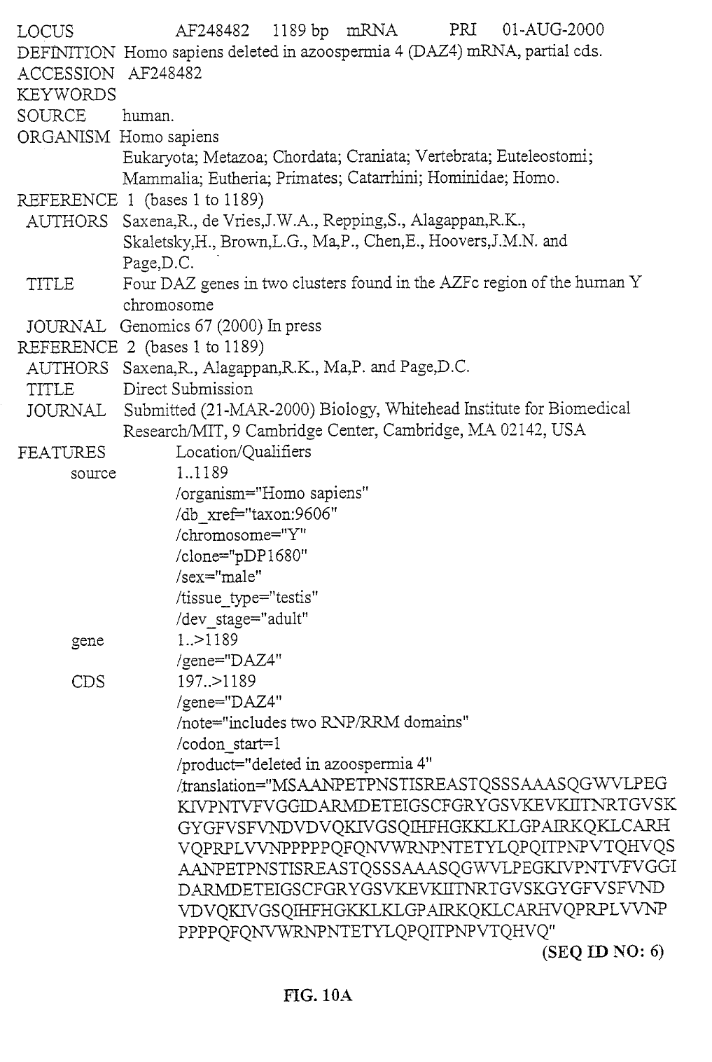

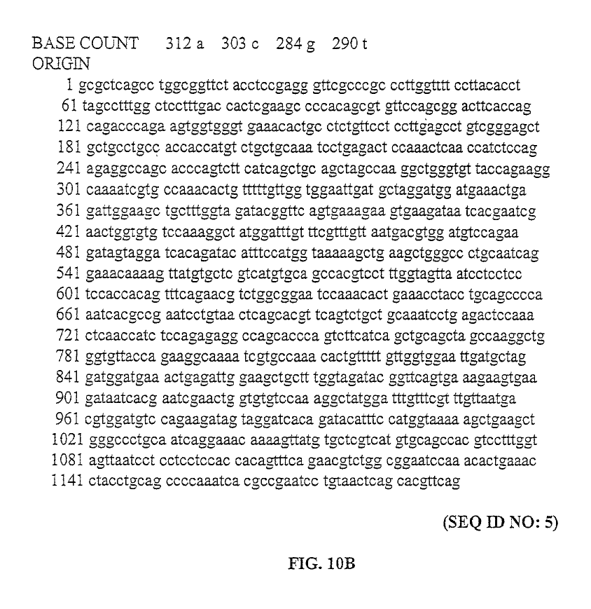

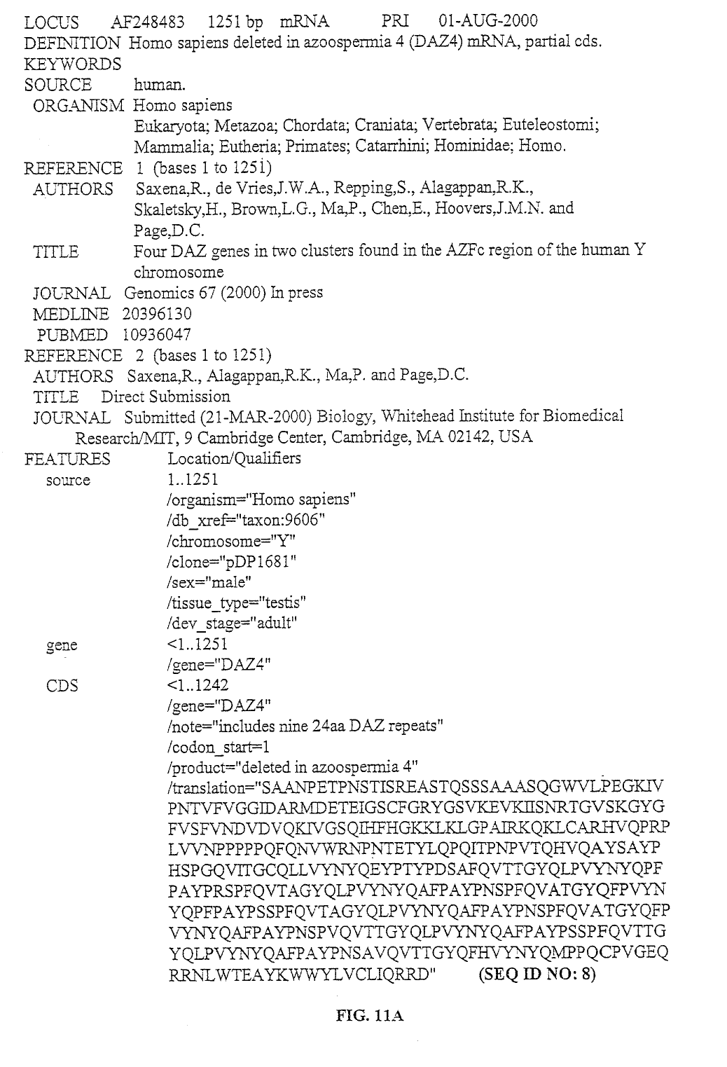

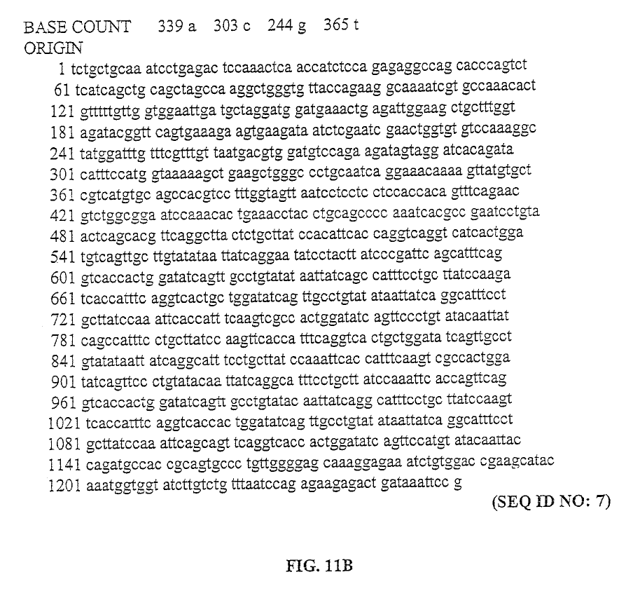

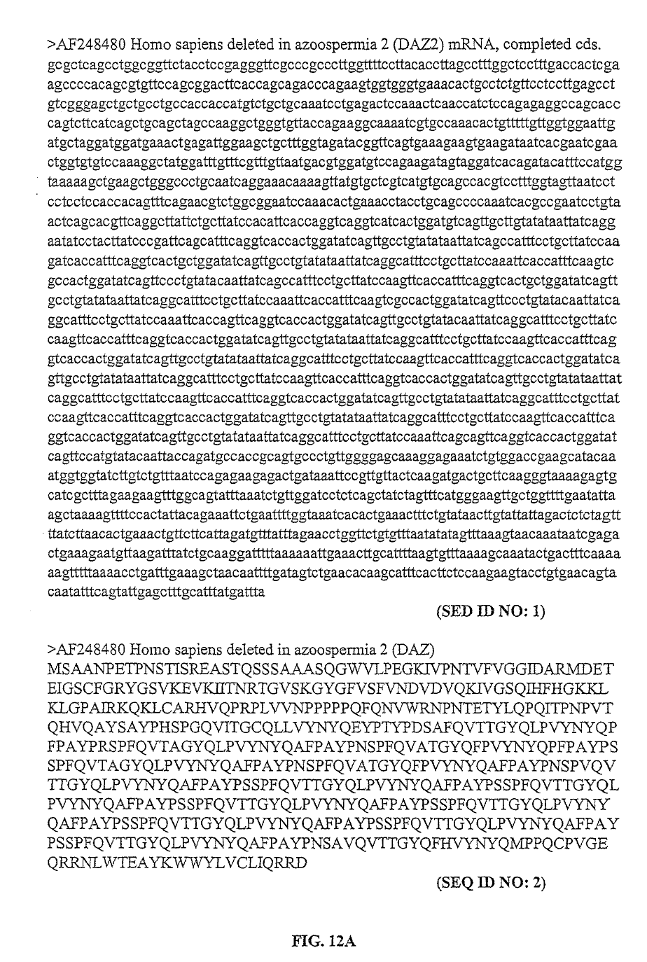

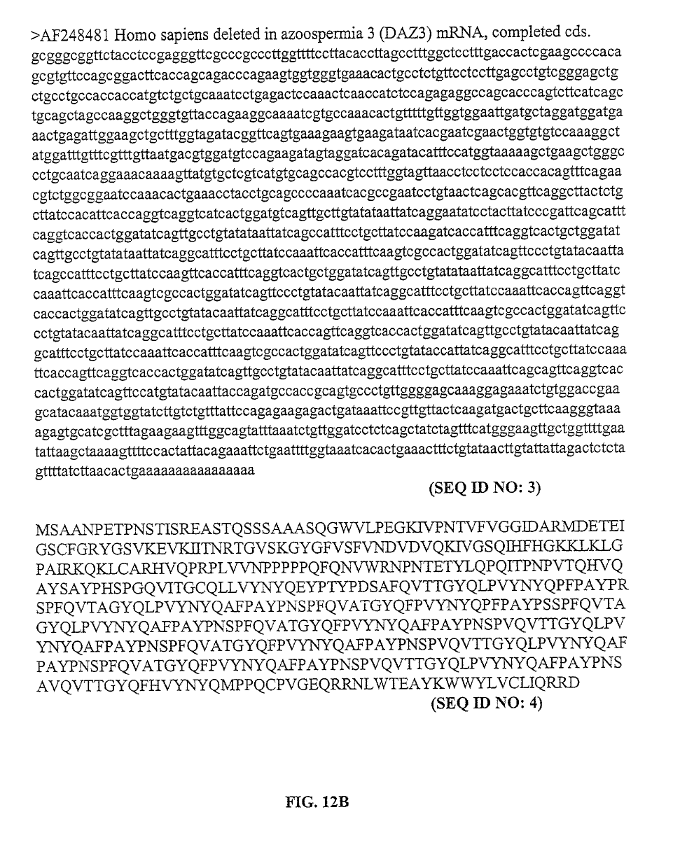

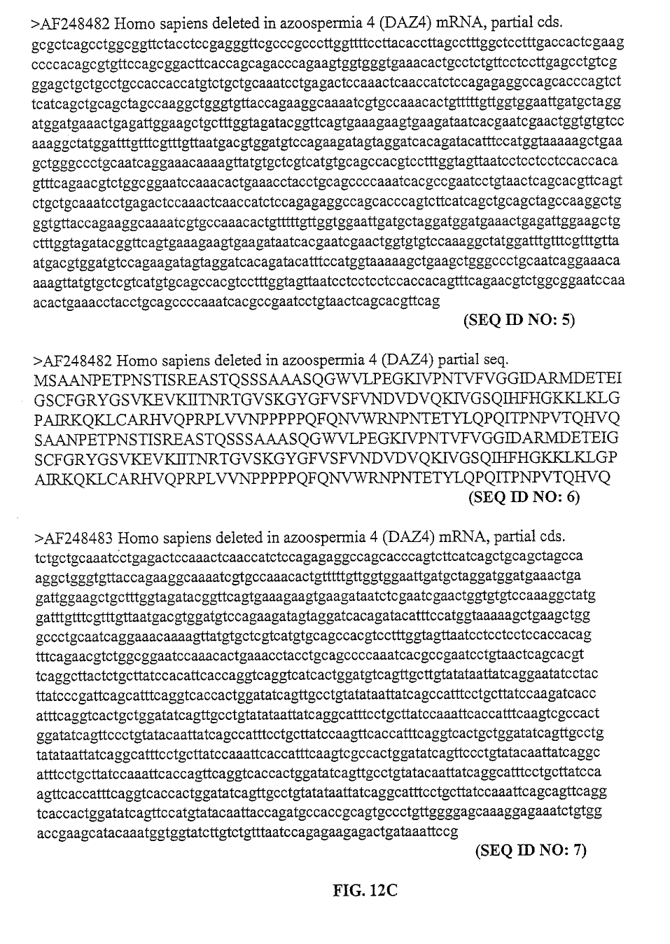

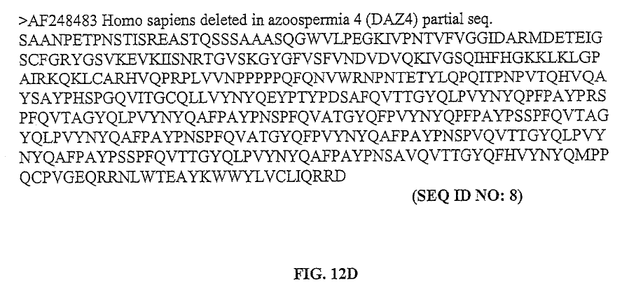

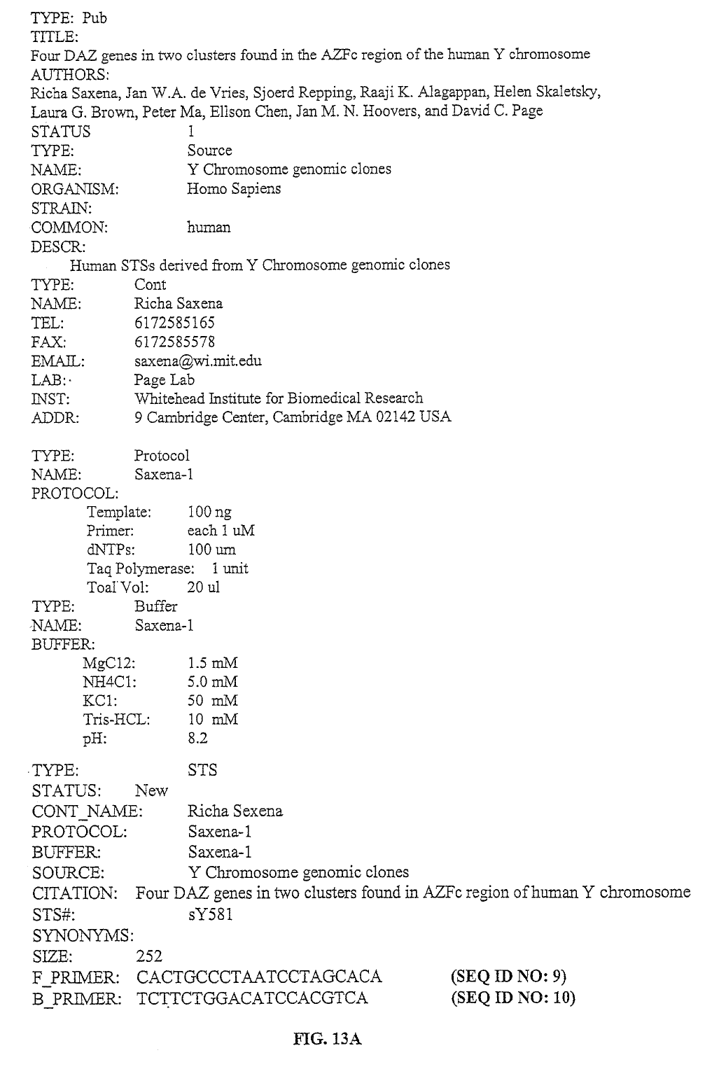

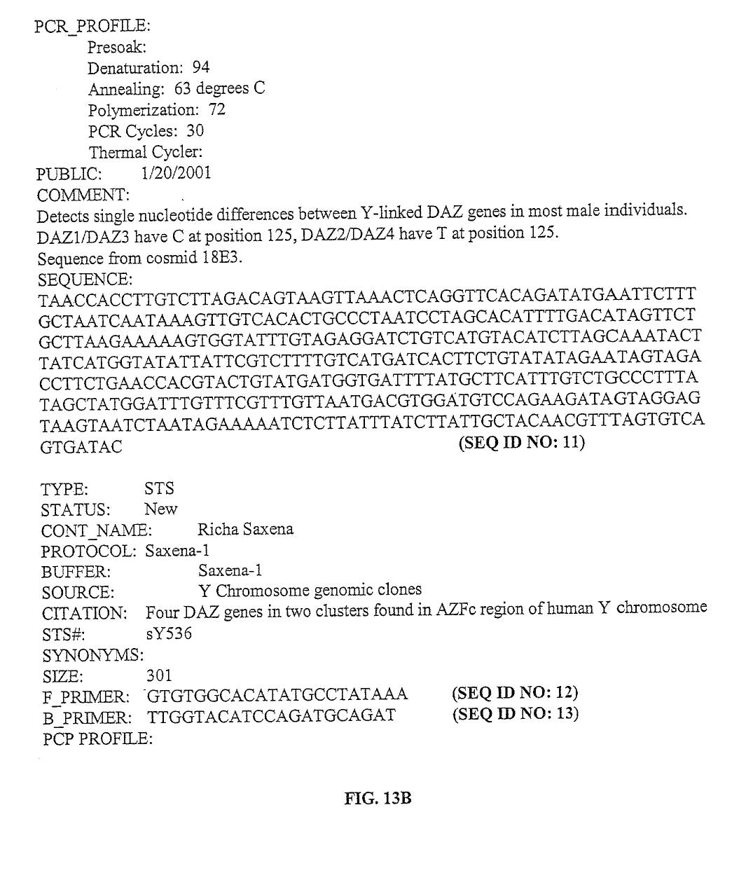

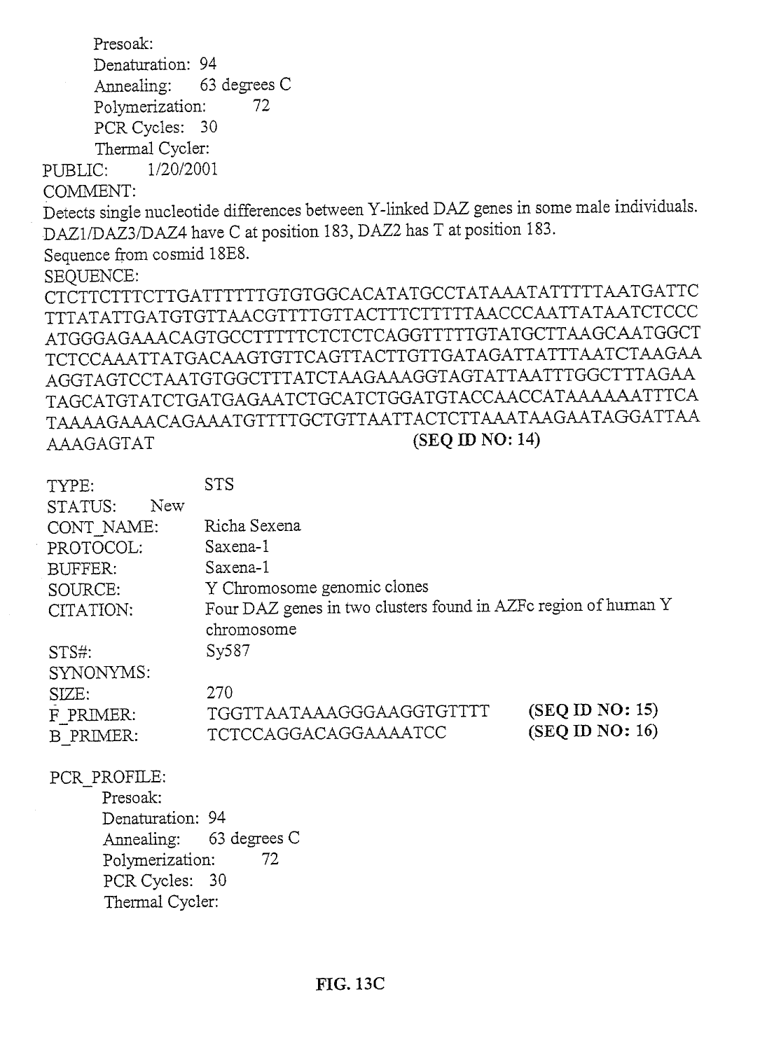

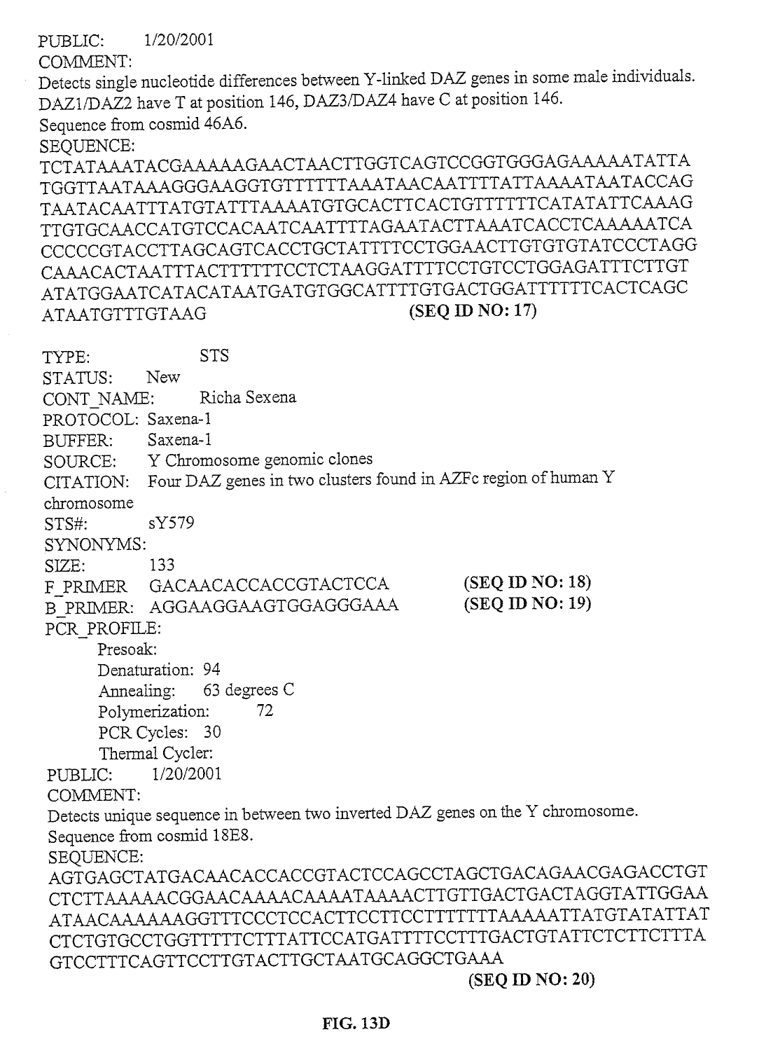

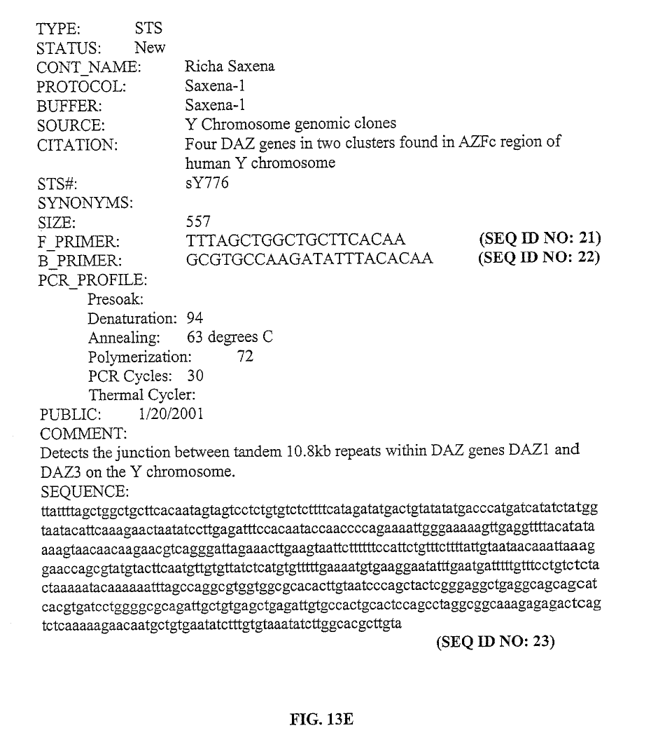

This nucleotide sequence analysis allowed the prediction of the primary structures of the DAZ proteins, which are depicted schematically, together with the autosomally encoded DAZL protein (Saxena, 1996), in FIG. 5. The large arrow is a 165-amino acid unit encompassing 82-amino acid RRM (RNA recognition motif). Smaller arrowheads are 24-amino acid units labeled according to the nomenclature of Yen et al., (1997). The C-terminal portion (open rectangle of the DAZL protein has no similarity to the C-terminal portions of the DAZ proteints. The 24-amino-acid units that are tandemly repeated in DAZ proteins show variability in sequence, as recognized previously (Reijo, 1995; Yen, 1997). To denote the distinct forms of the 24-amino-acid repeat (encoded by distinct forms of exon 7), the nomenclature (types “A, B, C, D, E, F, X, Y, Z”) suggested by Yen et al., (Yen, 1997) is used (FIG. 5).

Two features of the first full-length cDNA clone (pDP1678) enabled its assignment to DAZ2. In this cDNA clone, the 5′-most copy of the exon 7 (the first 72-nucleotide repeat) is of type “A”. In the DAZ2 genomic locus, the 5′-most copy of the exon (within a 2.4-kb genomic repeat) is also type “A”. No “A”-type copies of exon 7 were found anywhere in the DAZ1, DAZ3 or DAZ4 genomic loci. Second, the DAZ2 cDNA clone continued seven tandem “Y”-type copies of exon 7. At the genomic level, each “Y”-type 2.4-kb repeat contains a single Mlul site. An array of appropriately spaced Mlul sites is found in the DAZ2 genomic locus (FIG. 2). The DAZ2 cDNA sequence reported here is predicted to encode a 559-amino-acid protein with a molecular weight of 63K. Two previously reported cDNA clones—clone pDP1577 described by Reijo (1995), and clone E3 described by Yen (1997)— also appear to derive from DAZ3.

The third full-length cDNA clone (pDP1680/pDP1681) most likely derives from DAZ4, but the possibility that it derives from DAZ1 can not be excluded. This cDNA clone differs dramatically form the DAZ2 and DAZ3 clones in that it contains a tandem duplication of a 495-nucleotide (165-amino-acid) unit. This unit corresponds precisely to exons 2 through 6 and is predicted to encode an entire RRM (RNA recognition motif) domain. The tandem duplication of this 495-nucleotide unit within the cDNA corresponds well to the tandem duplication of the 10.8-kb unit in the DAZ4 genomic locus (FIG. 2). The putative DAZ4 cDNA sequence reported here is predicted to encode a 578-amino-acid with a molecular weight of 85K.

The fourth and fifth cDNA clones sequenced (pDP1575 and pDP1576) were incomplete at their 5′ ends, and they most likely derive from DAZ4, or possibly from DAZ1. Neither cDNA extended sufficiently 5′ to include exon 1 but both appeared to derive from transcripts in which exons 2 through 6 were (at least) duplicated, consistent with their deriving from either DAZ1 or DAZ4. Both cDNAs contained nine copies of exon 7 (BCDEFEXYZ), as also found in putative DAZ4 cDNA pDP1680, suggesting that these clones may be derived from DAZ4.

Partial sequence analysis of the remaining 12 DAZ cDNA clones revealed no additional classes of transcripts. Nonetheless, the existence of polymorphic or alternatively spliced forms cannot be ruled out.

The contents of all references, patents and published patent applications cited throughout this application are hereby incorporated by reference. While this invention has been particularly shown and described with references to preferred embodiments thereof, it will be understood by those skilled in the art that various changes in form and details may be made therein without departing from the scope of the invention encompassed by the appended claims.

### REFERENCES

Charlesworth, B. (1996). The evolution of chromosomal sex determination and dosage compensation. Curr. Biol. 6:149-162.

Chen, C., Su, Y., Baybayan, P., Siruno, A., Nagaraja, R., Mezzarella, R., Schiessinger, D., and Chen, E. Y. (1996). Ordered shotgun sequencing of a 135 kb Xq25 YAC containing ANT-2 and four possible genes, including three confirmed by EST matches. Nucleic Acids Res. 24:4034-4041.

Devine, S. E., Chissoe, S. L., Eby, Y., Wilson, R. K., and Boeke, J. D. (1997). A transposon-based strategy for sequencing repetitive DNA in eukaryotic genomes. Genome Res. 7:551-63.

Eberhart, C. G., Maines, J. Z., and Wasserman, S. A. (1996). Meiotic cell cycle requirement for a fly homologue of human Deleted in Azoospermia. Nature 381:783-785.

Elliott, D. J., Millar, M. R., Oghene, K., Ross, A., Kiesewetter, F., Pryor, J., Mcintyre, M., Hargreave, T. B., Saunders, P. T. K., Vogt, P. H., Chandley, A. C., and Cooke, H. (1997). Expression of RBM in the nuclei of human germ cells is dependent on a critical region of the Y chromosome long arm. Proc. Natl Acad. Sci. USA 94:3848-3853.

Fidlerova, H., Senger, G., Kost, M., Sanseau, P., and Sheer, D. (1994). Two simple procedures for releasing chromatin from routinely fixed cells for fluorescence in situ hybridization. Cytogenet. Cell Genet. 65:203-205.

Glaser, B., Hieri, T., Taylor, K., Schiebel, K., Zeitler, S., Papadopoullos, K., Rappold, G., and Schempp, W. (1997). High-resolution fluorescence in situ hybridization of human Y-linked genes on released chromatin. Chromosome Res. 5:23-30.

Glaser, B., Yen, P. H., and Schempp, W. (1998). Fibre-fluorescence in situ hybridization unravels apparently seven DAZ genes or pseudogenes clustered within a Y-chromosome region frequently deleted in azoospermic males. Chromosome Res. 6:481-486.

Gromoll, J., Weinbauer, G. F., Skaletsky, H., Schiatt, S., Rocchietti-March, M., Page, D. C., and Nieschlag, E. (1999). The Old World monkey DAZ (Deleted in AZoospermia) gene yields insights into the evolution of the DAZ gene cluster on the human Y chromosome. Hum. Mol. Genet. 8:2017-2024

Haaf, T., and Ward, D. C. (1995). Higher order nuclear structure in mammalian sperm revealed by in situ hybridization and extended chromatin fibers. Exp. Cell Res. 219:604-611.

Houston, D. W., and King, M. L. (2000). A critical role for XdazI, a germ plasm-localized RNA, in the differentiation of primordial germ cells in Xenopus. Development 127:447-456.

Kent-First, M., Muallem, A., Shultz, J., Pryor, J., Roberts, K., Nolten, W., Meisner, L., Chandley, A., Gouchy, G., Jorgensen, L., Havighurst, T., and Grosch, J. (1999). Defining regions of the Y-chromosome responsible for male infertility and identification of a fourth AZF region (AZFd) by Y-chromosome microdeietion detection. Mol. Reprod. Dev. 53:27-41.

Lahn, B. T., and Page, D. C. (1997). Functional coherence of the human Y chromosome. Science 278:675-680.

Ma, K., Sharkey A., Kirsch, S., Vogt, P., Keil, R., Hargreave, T. B., McBeath, S., and Chandley, A. C. (1992). Towards the molecular localisation of the AZF locus: Mapping of microdeletions in azoospermic men within 14 subintervals of interval 6 of the human Y chromosome. Hum. Mol. Genet. 1:29-33.

Manz, E., Schnieders, F., Muller Brechlin, A., and Schmidtke, J. (1993). TSPY-related sequences represent a microheterogeneous gene family organized as constitutive elements in DYZ5 tandem repeat units on the human Y chromosome. Genomics 17:726-731.

Menke, D. B., Mutter, G. L., and Page, D. C. (1997). Expression of DAZ, an azoospermia factor candidate, in human spermatogonia. Am. J. Hum. Genet. 60:237-241.

Ohno, S. (1967). Sex Chromosomes and Sex-linked Genes. Springer-Verlag, Berlin

Redeker, E., Hoovers, J. M., Alders, M., van Moorsel, C. J., Ivens, A. C., Gregory, S., Kaikin, L., Bliek, J., de Galan, L., and van den Bogaard, R. (1994). An integrated physical map of 210 markers assigned to the short arm of human chromosome 11. Genomics 21:538-550.

Reijo, R., Lee, T. Y., Salo, P., Alagappan, R., Brown, L. G., Rosenberg, M., Rozen, S., Jaffe, T., Strauss, D., Hovatta, O., de la Chapelle, A., Silber, S. and Page, D. C. (1995). Diverse spermatogenic defects in humans caused by Y chromosome deletions encompassing a novel RNA-binding protein gene. Nat. Genet 10:383-393.

Rice, W. R. (1994). Degeneration of a nonrecombining chromosome. Science 263:230-232.

Ruggiu, M., Speed, R., Taggart, M., McKay, S. J., Kilanowski, F., Saunders, P., Dorin, J., and Cooke, H. J. (1997). The mouse Dazla gene encodes a cytoplasmic protein essential for gametogenesis. Nature 389:73-77.

Saxena, R., Brown, L. G., Hawkins, T., Alagappan, R. K., Skaletsky, H., Reeve, M. P., Reijo, R., Rozen, S., Dinulos, M. B., Disteche, C. M., and Page, D. C. (1996). The DAZ gene cluster on the human Y chromosome arose from an autosomal gene that was transposed, repeatedly amplified and pruned. Nat. Genet 14:292-299.

Seboun, E., Barbaux, S., Bourgeron, T., Nishi, S., Algonik, A., Egashira, M., Nikkawa, N., Bishop, C., Fellous, M., McElreavey, K., and Kasahara, M. (1997) Gene sequence, localization, and evolutionary conservation of DAZLA, a candidate male sterility gene. Genomics 41:227-235

Shizuya, H. B., Birren, B., Kim, U. -J., Mancino, V., Slepak, T., Tachiiri, Y., and Simon, M. (1992). Cloning and stable maintenance of 300-kilobase-pair fragments of human DNA in Escherichia coli using an F-factor-based vector. Proc. Natl Acad. Sci. USA 89:8794-8797.

Silber, S. J. (1989). The relationship of abnormal semen parameters to male fertility. Hum. Reprod. 4:947-953.

Vogt, P. H., Edelmann, A., Kirsch, S., Henegariu, O., Hirschmann, P., Kiesewetter, F., Köhn, F. M., Schill, W. B., Farah, S., Ramos, C., Hartmann, M., Hartschuh, W., Meschede, D., Behre, H. M., Castel, A., Nieschlag, E., Weidner, W., Gröne, H. J., Jung, A., Engel, W., and Haidl, G. (1996). Human Y chromosome azoospermia factors (AZF) mapped to different subregions in Yq11. Hum. Mol Genet. 5:933-943.

Voilrath, D., Foote, S., Hilton, A., Brown, L. G., Beer-Romero, P., Bogan, J. S., and Page, D. C. (1992). The human Y chromosome: A 43-interval map based on naturally occurring deletions. Science 258:52-59.

Yen, P. H., Chai, N. N., and Salido, E. C. (1997). The human DAZ genes, a putative male infertility factor on the Y chromosome, are highly polymorphic in the DAZ repeat regions. Mamm. Genome 8:756-759.

Yen, P. H. (i 998). A long-range restriction map of deletion interval 6 of the human Y chromosome: A region frequently deleted in azoospermic males. Genomics 54:5-12.

What is claimed is:

1. An isolated nucleic acid molecule comprising a nucleotide sequence selected from the group consisting of:
a) SEQ ID NOS: 1, 3, 5, 7 and 9-23; and
b) the complement of SEQ ID NOS: 1, 3, 5, 7 and 9-23.

2. An isolated nucleic acid molecule according to claim 1, which is expressed in testicular germ cells.

3. An isolated nucleic acid molecule according to claim 1, which is DNA

4. An isolated nucleic acid molecule according to claim 1, which is RNA.

5. An isolated nucleic acid molecule selected from the group consisting of nucleotide 197 to nucleotide 1873 of SEQ ID NO: 1, nucleotide 189 to nucleotide 1649 of SEQ ID NO: 3, and nucleotide 1 to nucleotide 1242 of SEQ ID NO: 5.

6. An isolated nucleic acid molecule consisting of a nucleotide sequence selected from the group consisting of:
a) SEQ ID NOS: 1, 3, 5, 7 and 9-23; and
b) the complement of SEQ ID NOS: 1, 3, 5, 7 and 9-23.

7. A portion of an isolated nucleic acid molecule consisting of a nucleotide sequence selected from the group consisting of:
a) SEQ ID NOS: 1, 3, 5, 7 and 9-23; and
b) the complement of SEQ ID NOS: 1, 3, 5, 7 and 9-23;
wherein the portion is at least about 10 contiguous nucleotides in length.

8. A nucleic acid construct comprising the isolated nucleic acid molecule of claim 1.

9. The nucleic acid construct of claim 8, wherein the isolated nucleic acid molecule is operatively linked to a regulatory sequence.

10. A recombinant host cell comprising the isolated nucleic acid molecule of claim 1.

11. The recombinant host cell of claim 10, wherein said cell is selected from the group consisting of bacterial cells, fungal cells, plant cells, insect cells and mammalian cells.

12. A method for preparing a polypeptide encoded by an isolated nucleic acid molecule, comprising culturing the recombinant host cell of claim 10.

13. A method for assaying for the presence of a DAZ polypeptide in a sample, comprising contacting said sample with an agent which specifically detects the DAZ polypeptide.

14. A polypeptide encoded by an isolated nucleic acid molecule according to claim 1.

15. An isolated polypeptide comprising an amino acid sequence selected from the group consisting of SEQ ID NOS: 2, 4, 6 and 8.

16. An antibody, or an antigen-binding fragment thereof, which selectively binds to the polypeptide encoded by an isolated nucleic acid molecule according to claim 1, or to a portion of said polypeptide.

17. An isolated nucleic acid molecule comprising a nucleotide sequence which is at least about 60% identical to a nucleotide sequence selected from the group consisting of:
a) SEQ ID NOS: 1, 3, 5, 7 and 9-23; and
b) the complement of SEQ ID NOS: 1, 3, 5, 7 and 9-23.

18. An isolated nucleic acid molecule which hybridizes under high stringency conditions to a nucleotide sequence selected from the group consisting of:
a) SEQ ID NOS: 1, 3, 5, 7 and 9-23; and
b) the complement of SEQ ID NOS: 1, 3, 5, 7 and 9-23.

19. An isolated nucleic acid molecule encoding SEQ ID NO: 2.

20. An isolated nucleic acid molecule encoding SEQ ID NO: 4.

21. An isolated nucleic acid molecule encoding an amino acid sequence selected from the group consisting of SEQ ID NO: 6 and SEQ ID NO: 8.

22. A method for assaying the presence of a DAZ nucleic acid molecule in a sample, comprising;
a) contacting said sample with a nucleotide sequence selected from the group consisting of:
i) SEQ ID NOS: 1, 3, 5, 7 and 9-23;
ii) the complement of SEQ ID NOS: 1, 3, 5, 7 and 9-23;
iii) a portion of any one of SEQ ID NOS: 1, 3, 5, 7 and 9-23 which is at least 10 contiguous nucleotides in length; and
iv) a portion of the complement of any one of SEQ ID NOS: 1, 3, 5, 7 and 9-23, which is at least 10 contiguous nucleotides in length, under conditions appropriate for selective hybridization, such that the nucleotide sequence binds to complementary nucleic acid molecule in the sample, if present; and
b) detecting the hybridized nucleotide sequence.

23. The method of claim 22, wherein the nucleotide sequence is selected from the group consisting of: nucleotide 197 to nucleotide 1873 of SEQ ID NO: 1, nucleotide 189 to nucleotide 1649 of SEQ ID NO: 3, and nucleotide 1 to nucleotide 1242 of SEQ ID NO: 5.

24. The method of claim 22, wherein the sample comprises human chromosomal DNA.

25. The method of claim 22, wherein the sample comprises human mRNA or cDNA.

26. A method for distinguishing a DAZ gene of interest from other DAZ genes by detecting sequence family variants comprising;
a) conducting at least one amplification reaction to amplify at least one region of a DAZ gene;
b) digesting the amplified product with a restriction endonuclease; and
c) detecting products of the digestion, wherein the products of the digestion distinguishes the DAZ gene of interest from other DAZ genes.

27. The method of claim 26, wherein amplification is conducted by polymerase chain reaction using one or more primers selected from the group consisting of SEQ ID NOS: 9, 10, 12, 13, 15, 16, 18, 19, 21, 22, 23, 24 and combinations thereof.

28. The method of claim 26, wherein the amplified product is digested with at least one restriction enzyme selected from the group consisting of Sau3A, TaqI, Dral and combinations thereof.

29. An isolated polypeptide comprising an amino acid selected from the group consisting of SEQ ID NOS: 2, 4, 6 and 8.

30. A method for analyzing a sample for the presence of DAZ gene product, comprising;
a) contacting said sample with an antibody specific for a polypeptide selected from the group consisting of: SEQ ID NOS: 2, 4, 6 and 8; under conditions appropriate for the antibody to bind said polypeptide, if present, in the sample; and
b) detecting the bound antibody

31. The method of claim 30, wherein the sample is derived from testes.

32. A method for identifying an agent that alters the activity of a DAZ polypeptide comprising contacting a polypeptide, or functional fragment thereof, selected from the group consisting of:
(a) SEQ ID NO: 2;
(b) SEQ ID NO: 4;
(c) SEQ ID NO: 6;
(d) SEQ ID NO: 8;
(e) an amino acid sequence encoded by SEQ ID NO: 1;
(f) an amino acid sequence encoded by SEQ ID NO: 3;
(g) an amino acid sequence encoded by SEQ ID NO: 5;
(h) an amino acid sequence encoded by SEQ ID NO: 7; and
(i) functional fragments of (a)-(h)
with an agent to be tested, determining the level of activity of the polypeptide in the presence of the agent, and comparing said level of activity with the level of activity of the polypeptide in the absence of the agent, wherein a statistically-significant change in activity is indicative that the agent alters the activity of the polypeptide.

33. The method according to claim 32 wherein the activity is binding to RNA.

34. A method of treating an individual having a disorder associated with reduced DAZ activity comprising administering a therapeutically-effective amount of a DAZ agonist.

35. The method according to claim 34 wherein the disorder is infertility

36. A method of reducing the fertility of an individual comprising administering a therapeutically-effective amount of a DAZ antagonist.


SEQUENCE LISTING (25 sequences)

SEQ ID NO: 1 - DNA, 2408 bp, Homo sapiens
  CDS (197)...(1873)
gcgctcagcctggcggttctacctccgagggttcgcccgcccttggttttccttacaccttagcctttggctcctttgaccactcgaagccccacagcgtgttccagcggacttcaccagcagacccagaagtggtgggtgaaacactgcctctgttcctccttgagcctgtcgggagctgctgcctgccaccaccatgtctgctgcaaatcctgagactccaaactcaaccatctccagagaggccagcacccagtcttcatcagctgcagctagccaaggctgggtgttaccagaaggcaaaatcgtgccaaacactgtttttgttggtggaattgatgctaggatggatgaaactgagattggaagctgctttggtagatacggttcagtgaaagaagtgaagataatcacgaatcgaactggtgtgtccaaaggctatggatttgtttcgtttgttaatgacgtggatgtccagaagatagtaggatcacagatacatttccatggtaaaaagctgaagctgggccctgcaatcaggaaacaaaagttatgtgctcgtcatgtgcagccacgtcctttggtagttaatcctcctcctccaccacagtttcagaacgtctggcggaatccaaacactgaaacctacctgcagccccaaatcacgccgaatcctgtaactcagcacgttcaggcttattctgcttatccacattcaccaggtcaggtcatcactggatgtcagttgcttgtatataattatcaggaatatcctacttatcccgattcagcatttcaggtcaccactggatatcagttgcctgtatataattatcagccatttcctgcttatccaagatcaccatttcaggtcactgctggatatcagttgcctgtatataattatcaggcatttcctgcttatccaaattcaccatttcaagtcgccactggatatcagttccctgtatacaattatcagccatttcctgcttatccaagttcaccatttcaggtcactgctggatatcagttgcctgtatataattatcaggcatttcctgcttatccaaattcaccatttcaagtcgccactggatatcagttccctgtatacaattatcaggcatttcctgcttatccaaattcaccagttcaggtcaccactggatatcagttgcctgtatacaattatcaggcatttcctgcttatccaagttcaccatttcaggtcaccactggatatcagttgcctgtatataattatcaggcatttcctgcttatccaagttcaccatttcaggtcaccactggatatcagttgcctgtatataattatcaggcatttcctgcttatccaagttcaccatttcaggtcaccactggatatcagttgcctgtatataattatcaggcatttcctgcttatccaagttcaccatttcaggtcaccactggatatcagttgcctgtatataattatcaggcatttcctgcttatccaagttcaccatttcaggtcaccactggatatcagttgcctgtatataattatcaggcatttcctgcttatccaagttcaccatttcaggtcaccactggatatcagttgcctgtatataattatcaggcatttcctgcttatccaagttcaccatttcaggtcaccactggatatcagttgcctgtatataattatcaggcatttcctgcttatccaaattcagcagttcaggtcaccactggatatcagttccatgtatacaattaccagatgccaccgcagtgccctgttggggagcaaaggagaaatctgtggaccgaagcatacaaatggtggtatcttgtctgtttaatccagagaagagactgataaattccgttgttactcaagatgactgcttcaagggtaaaagagtgcatcgctttagaagaagtttggcagtatttaaatctgttggatcctctcagctatctagtttcatgggaagttgctggttttgaatattaagctaaaagttttccactattacagaaattctgaattttggtaaatcacactgaaactttctgtataacttgtattattagactctctagttttatcttaacactgaaactgttcttcattagatgtttatttagaacctggttctgtgtttaatatatagtttaaagtaacaaataatcgagactgaaagaatgttaagatttatctgcaaggatttttaaaaaattgaaacttgcattttaagtgtttaaaagcaaatactgactttcaaaaaagtttttaaaacctgatttgaaagctaacaattttgatagtctgaacacaagcatttcacttctccaagaagtacctgtgaacagtacaatatttcagtattgagctttgcatttatgattta

SEQ ID NO: 2 - protein, 558 aa, Homo sapiens
MSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQAYSAYPHSPGQVITGCQLLVYNYQEYPTYPDSAFQVTTGYQLPVYNYQPFPAYPRSPFQVTAGYQLPVYNYQAFPAYPNSPFQVATGYQFPVYNYQPFPAYPSSPFQVTAGYQLPVYNYQAFPAYPNSPFQVATGYQFPVYNYQAFPAYPNSPVQVTTGYQLPVYNYQAFPAYPSSPFQVTTGYQLPVYNYQAFPAYPSSPFQVTTGYQLPVYNYQAFPAYPSSPFQVTTGYQLPVYNYQAFPAYPSSPFQVTTGYQLPVYNYQAFPAYPSSPFQVTTGYQLPVYNYQAFPAYPSSPFQVTTGYQLPVYNYQAFPAYPSSPFQVTTGYQLPVYNYQAFPAYPNSAVQVTTGYQFHVYNYQMPPQCPVGEQRRNLWTEAYKWWYLVCLIQRRD

SEQ ID NO: 3 - DNA, 1908 bp, Homo sapiens
  CDS (189)...(1649)
gcgggcggttctacctccgagggttcgcccgcccttggttttccttacaccttagcctttggctcctttgaccactcgaagccccacagcgtgttccagcggacttcaccagcagacccagaagtggtgggtgaaacactgcctctgttcctccttgagcctgtcgggagctgctgcctgccaccaccatgtctgctgcaaatcctgagactccaaactcaaccatctccagagaggccagcacccagtcttcatcagctgcagctagccaaggctgggtgttaccagaaggcaaaatcgtgccaaacactgtttttgttggtggaattgatgctaggatggatgaaactgagattggaagctgctttggtagatacggttcagtgaaagaagtgaagataatcacgaatcgaactggtgtgtccaaaggctatggatttgtttcgtttgttaatgacgtggatgtccagaagatagtaggatcacagatacatttccatggtaaaaagctgaagctgggccctgcaatcaggaaacaaaagttatgtgctcgtcatgtgcagccacgtcctttggtagttaatcctcctcctccaccacagtttcagaacgtctggcggaatccaaacactgaaacctacctgcagccccaaatcacgccgaatcctgtaactcagcacgttcaggcttactctgcttatccacattcaccaggtcaggtcatcactggatgtcagttgcttgtatataattatcaggaatatcctacttatcccgattcagcatttcaggtcaccactggatatcagttgcctgtatataattatcagccatttcctgcttatccaagatcaccatttcaggtcactgctggatatcagttgcctgtatataattatcaggcatttcctgcttatccaaattcaccatttcaagtcgccactggatatcagttccctgtatacaattatcagccatttcctgcttatccaagttcaccatttcaggtcactgctggatatcagttgcctgtatataattatcaggcatttcctgcttatccaaattcaccatttcaagtcgccactggatatcagttccctgtatacaattatcaggcatttcctgcttatccaaattcaccagttcaggtcaccactggatatcagttgcctgtatacaattatcaggcatttcctgcttatccaaattcaccatttcaagtcgccactggatatcagttccctgtatacaattatcaggcatttcctgcttatccaaattcaccagttcaggtcaccactggatatcagttgcctgtatacaattatcaggcatttcctgcttatccaaattcaccatttcaagtcgccactggatatcagttccctgtataccattatcaggcatttcctgcttatccaaattcaccagttcaggtcaccactggatatcagttgcctgtatacaattatcaggcatttcctgcttatccaaattcagcagttcaggtcaccactggatatcagttccatgtatacaattaccagatgccaccgcagtgccctgttggggagcaaaggagaaatctgtggaccgaagcatacaaatggtggtatcttgtctgtttattccagagaagagactgataaattccgttgttactcaagatgactgcttcaagggtaaaagagtgcatcgctttagaagaagtttggcagtatttaaatctgttggatcctctcagctatctagtttcatgggaagttgctggttttgaatattaagctaaaagttttccactattacagaaattctgaattttggtaaatcacactgaaactttctgtataacttgtattattagactctctagttttatcttaacactgaaaaaaaaaaaaaaaa

SEQ ID NO: 4 - protein, 486 aa, Homo sapiens
MSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQAYSAYPHSPGQVITGCQLLVYNYQEYPTYPDSAFQVTTGYQLPVYNYQPFPAYPRSPFQVTAGYQLPVYNYQAFPAYPNSPFQVATGYQFPVYNYQPFPAYPSSPFQVTAGYQLPVYNYQAFPAYPNSPFQVATGYQFPVYNYQAFPAYPNSPVQVTTGYQLPVYNYQAFPAYPNSPFQVATGYQFPVYNYQAFPAYPNSPVQVTTGYQLPVYNYQAFPAYPNSPFQVATGYQFPVYHYQAFPAYPNSPVQVTTGYQLPVYNYQAFPAYPNSAVQVTTGYQFHVYNYQMPPQCPVGEQRRNLWTEAYKWWYLVCLFQRRD

SEQ ID NO: 5 - DNA, 1189 bp, Homo sapiens
  CDS (197)...(1189)
gcgctcagcctggcggttctacctccgagggttcgcccgcccttggttttccttacaccttagcctttggctcctttgaccactcgaagccccacagcgtgttccagcggacttcaccagcagacccagaagtggtgggtgaaacactgcctctgttcctccttgagcctgtcgggagctgctgcctgccaccaccatgtctgctgcaaatcctgagactccaaactcaaccatctccagagaggccagcacccagtcttcatcagctgcagctagccaaggctgggtgttaccagaaggcaaaatcgtgccaaacactgtttttgttggtggaattgatgctaggatggatgaaactgagattggaagctgctttggtagatacggttcagtgaaagaagtgaagataatcacgaatcgaactggtgtgtccaaaggctatggatttgtttcgtttgttaatgacgtggatgtccagaagatagtaggatcacagatacatttccatggtaaaaagctgaagctgggccctgcaatcaggaaacaaaagttatgtgctcgtcatgtgcagccacgtcctttggtagttaatcctcctcctccaccacagtttcagaacgtctggcggaatccaaacactgaaacctacctgcagccccaaatcacgccgaatcctgtaactcagcacgttcagtctgctgcaaatcctgagactccaaactcaaccatctccagagaggccagcacccagtcttcatcagctgcagctagccaaggctgggtgttaccagaaggcaaaatcgtgccaaacactgtttttgttggtggaattgatgctaggatggatgaaactgagattggaagctgctttggtagatacggttcagtgaaagaagtgaagataatcacgaatcgaactggtgtgtccaaaggctatggatttgtttcgtttgttaatgacgtggatgtccagaagatagtaggatcacagatacatttccatggtaaaaagctgaagctgggccctgcaatcaggaaacaaaagttatgtgctcgtcatgtgcagccacgtcctttggtagttaatcctcctcctccaccacagtttcagaacgtctggcggaatccaaacactgaaacctacctgcagccccaaatcacgccgaatcctgtaactcagcacgttcag

SEQ ID NO: 6 - protein, 331 aa, Homo sapiens
MSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQ

SEQ ID NO: 7 - DNA, 1251 bp, Homo sapiens
  CDS (1)...(1242)
tctgctgcaaatcctgagactccaaactcaaccatctccagagaggccagcacccagtcttcatcagctgcagctagccaaggctgggtgttaccagaaggcaaaatcgtgccaaacactgtttttgttggtggaattgatgctaggatggatgaaactgagattggaagctgctttggtagatacggttcagtgaaagaagtgaagataatctcgaatcgaactggtgtgtccaaaggctatggatttgtttcgtttgttaatgacgtggatgtccagaagatagtaggatcacagatacatttccatggtaaaaagctgaagctgggccctgcaatcaggaaacaaaagttatgtgctcgtcatgtgcagccacgtcctttggtagttaatcctcctcctccaccacagtttcagaacgtctggcggaatccaaacactgaaacctacctgcagccccaaatcacgccgaatcctgtaactcagcacgttcaggcttactctgcttatccacattcaccaggtcaggtcatcactggatgtcagttgcttgtatataattatcaggaatatcctacttatcccgattcagcatttcaggtcaccactggatatcagttgcctgtatataattatcagccatttcctgcttatccaagatcaccatttcaggtcactgctggatatcagttgcctgtatataattatcaggcatttcctgcttatccaaattcaccatttcaagtcgccactggatatcagttccctgtatacaattatcagccatttcctgcttatccaagttcaccatttcaggtcactgctggatatcagttgcctgtatataattatcaggcatttcctgcttatccaaattcaccatttcaagtcgccactggatatcagttccctgtatacaattatcaggcatttcctgcttatccaaattcaccagttcaggtcaccactggatatcagttgcctgtatacaattatcaggcatttcctgcttatccaagttcaccatttcaggtcaccactggatatcagttgcctgtatataattatcaggcatttcctgcttatccaaattcagcagttcaggtcaccactggatatcagttccatgtatacaattaccagatgccaccgcagtgccctgttggggagcaaaggagaaatctgtggaccgaagcatacaaatggtggtatcttgtctgtttaatccagagaagagactgataaattccg

SEQ ID NO: 8 - protein, 413 aa, Homo sapiens
SAANPETPNSTISREASTQSSSAAASQGWVLPEGKIVPNTVFVGGIDARMDETEIGSCFGRYGSVKEVKIISNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQAYSAYPHSPGQVITGCQLLVYNYQEYPTYPDSAFQVTTGYQLPVYNYQPFPAYPRSPFQVTAGYQLPVYNYQAFPAYPNSPFQVATGYQFPVYNYQPFPAYPSSPFQVTAGYQLPVYNYQAFPAYPNSPFQVATGYQFPVYNYQAFPAYPNSPVQVTTGYQLPVYNYQAFPAYPSSPFQVTTGYQLPVYNYQAFPAYPNSAVQVTTGYQFHVYNYQMPPQCPVGEQRRNLWTEAYKWWYLVCLIQRRD

SEQ ID NO: 9 - DNA, 21 bp, Homo sapiens
cactgccctaatcctagcaca

SEQ ID NO: 10 - DNA, 20 bp, Homo sapiens
tcttctggacatccacgtca

SEQ ID NO: 11 - DNA, 401 bp, Homo sapiens
taaccaccttgtcttagacagtaagttaaactcaggttcacagatatgaattctttgctaatcaataaagttgtcacactgccctaatcctagcacattttgacatagttctgcttaagaaaaagtggtatttgtagaggatctgtcatgtacatcttagcaaatacttatcatggtatattattcgtcttttgtcatgatcacttctgtatatagaatagtagaccttctgaaccacgtactgtatgatggtgattttatgcttcatttgtctgcctttatagctatggatttgtttcgtttgttaatgacgtggatgtccagaagatagtaggagtaagtaatctaatagaaaaatctcttatttatcttattgctacaacgtttagtgtcagtgatac

SEQ ID NO: 12 - DNA, 22 bp, Homo sapiens
gtgtggcacatatgcctataaa

SEQ ID NO: 13 - DNA, 21 bp, Homo sapiens
ttggtacatccagatgcagat

SEQ ID NO: 14 - DNA, 400 bp, Homo sapiens
ctcttctttcttgattttttgtgtggcacatatgcctataaatatttttaatgattctttatattgatgtgttaacgttttgttactttctttttaacccaattataatctcccatgggagaaacagtgcctttttctctctcaggtttttgtatgcttaagcaatggcttctccaaattatgacaagtgttcagttacttgttgatagattatttaatctaagaaaggtagtcctaatgtggctttatctaagaaaggtagtattaatttggctttagaatagcatgtatctgatgagaatctgcatctggatgtaccaaccataaaaaatttcataaaagaaacagaaatgttttgctgttaattactcttaaataagaataggattaaaaagagtat

SEQ ID NO: 15 - DNA, 24 bp, Homo sapiens
tggttaataaagggaaggtgtttt

SEQ ID NO: 16 - DNA, 20 bp, Homo sapiens
tctccaggacaggaaaatcc

SEQ ID NO: 17 - DNA, 401 bp, Homo sapiens
tctataaatacgaaaaagaactaacttggtcagtccggtgggagaaaaatattatggttaataaagggaaggtgttttttaaataacaattttattaaaataataccagtaatacaatttatgtatttaaaatgtgcacttcactgttttttcatatattcaaagttgtgcaaccatgtccacaatcaattttagaatacttaaatcacctcaaaaatcacccccgtaccttagcagtcacctgctattttcctggaacttgtgtgtatccctaggcaaacactaatttacttttttcctctaaggattttcctgtcctggagatttcttgtatatggaatcatacataatgatgtggcattttgtgactggattttttcactcagcataatgtttgtaag

SEQ ID NO: 18 - DNA, 20 bp, Homo sapiens
gacaacaccaccgtactcca

SEQ ID NO: 19 - DNA, 20 bp, Homo sapiens
aggaaggaagtggagggaaa

SEQ ID NO: 20 - DNA, 264 bp, Homo sapiens
agtgagctatgacaacaccaccgtactccagcctagctgacagaacgagacctgtctcttaaaaacggaacaaaacaaaataaaacttgttgactgactaggtattggaaataacaaaaaaggtttccctccacttccttcctttttttaaaaattatgtatattatctctgtgcctggtttttctttattccatgattttcctttgactgtattctcttctttagtcctttcagttccttgtacttgctaatgcaggctgaaa

SEQ ID NO: 21 - DNA, 20 bp, Homo sapiens
tttagctggctgcttcacaa

SEQ ID NO: 22 - DNA, 22 bp, Homo sapiens
gcgtgccaagatatttacacaa

SEQ ID NO: 23 - DNA, 566 bp, Homo sapiens
ttattttagctggctgcttcacaatagtagtcctctgtgtctcttttcatagatatgactgtatatatgacccatgatcatatctatggtaatacattcaaagaactaatatccttgagatttccacaataccaaccccagaaaattgggaaaaagttgaggttttacatataaaagtaacaacaagaacgtcagggattagaaacttgaagtaattcttttttccattctgtttcttttattgtaataacaaattaaaggaaccagcgtatgtacttcaatgttgtgttatctcatgtgtttttgaaaatgtgaaggaatatttgaatgatttttgtttcctgtctctactaaaaatacaaaaaatttagccaggcgtggtggcgcacacttgtaatcccagctactcgggaggctgaggcagcagcatcacgtgatcctggggcgcagattgctgtgagctgagattgtgccactgcactccagcctaggcggcaaagagagactcagtctcaaaaagaacaatgctgtgaatatctttgtgtaaatatcttggcacgcttgta

SEQ ID NO: 24 - DNA, 25 bp, Artificial Sequence
  Primer
caggacattggatgctgagaattcg

SEQ ID NO: 25 - DNA, 23 bp, Artificial Sequence
  Primer
caggagccgtctatcctgcttgc